(19) United States
(12) Patent Application Publication
(10) Pub. No.: US 2022/0252611 A1
(43) Pub. Date: Aug. 11, 2022

(54) METHODS FOR USING MASS SPECTROSCOPY IN MULTIPLEX TARGET EVALUATIONS

(71) Applicant: EUROFINS CEREP, Celle-L'Evescault (FR)

(72) Inventors: Manilduth RAMNATH, Celle-L'Evescault (FR); Benoît FOUCHAQ, Poitiers (FR); Jérôme LAPARRE, Celle-L'Evescault (FR)

(73) Assignee: EUROFINS CEREP, Celle-L'Evescault (FR)

(21) Appl. No.: 17/691,117

(22) Filed: Mar. 9, 2022

Related U.S. Application Data
(63) Continuation of application No. PCT/EP2020/075250, filed on Sep. 9, 2020.

(30) Foreign Application Priority Data
Sep. 13, 2019 (EP) 19306104.1
Sep. 16, 2019 (EP) 19306110.8

(51) Int. Cl. G01N 33/68; G01N 30/72; G01N 33/94

(52) U.S. Cl. CPC G01N 33/6845; G01N 30/7266; G01N 2030/027; G01N 33/94; G01N 33/6848

(57) ABSTRACT

Provided are multiplexed methods for characterizing binding of a test compound to different receptor target molecules using mass spectroscopy techniques. The methods employ receptor molecules that have different functions or found in different tissues, such as cerebral cortex, cerebellum, ventricular and hepatic membrane preparations. The methods enable determination of undesirable off-target binding of a test compound. The methods comprise incubation of a heterologous mixture of different receptor target molecules with ligands (known binders), and a test compound. Various wells contain different amounts of molecules for use in construction of concentration curves. Next, unbound ligands are separated from the well contents. Next, ligands that were bound to the receptors are separated. An LC/ESI-MS/MS method may be used to reduce irrelevant mass spectroscopy peaks. Binding of the test compound to a desired receptor target molecule is compared to binding of the test compound to other receptor target molecules, i.e., off-target binding.

### CROSS REFERENCE TO RELATED APPLICATIONS

This application claims priority to Provisional Application Serial No. EP19306104 filed Sep. 13, 2019, and Provisional Application Serial No. EP19306110 filed Sep. 16, 2019, which are hereby incorporated by reference in its entirety.

### FIELD OF THE INVENTION

The present methods relate to the characterization of the binding of various compounds to target molecules, using a label free technology such as mass spectrometry (MS). They further relate to evaluating the affinity of ligands to a specific receptor target molecule.

### BACKGROUND

Presented below is background information on certain aspects of the present invention as they may relate to technical features referred to in the detailed description, but not necessarily described in detail. That is, individual parts or methods used in the present invention may be described in greater detail in the documents discussed below, which materials may provide further guidance to those skilled in the art for making or using certain aspects of the present invention as claimed. Such documents are hereby incorporated by reference into the present application. The discussion below should not be construed as an admission as to the relevance of the information to any claims herein or the prior art effect of the material described.

### SPECIFIC PATENTS AND PUBLICATIONS

Wanner et al., WO 2002095403 (U.S. Pat. No. 7,074,334), “Method for determining the binding behavior of ligands which specifically bind to target molecules,” discloses The invention relating to a method for determining the binding behavior of ligands which specifically bind to target molecules at least one binding site, whereby the markers are present in a native form and the concentrations K4 and K5 or the quantities M2 and M1 are determined by mass spectrometry.

Wanner et al. US Publication 2006/0201886, “Method for determining the binding behavior of ligands which specifically bind to target molecules,” discloses a method for determining the binding behavior of ligands which specifically bind to target molecules at least one binding site. The markers are present in a native form, and the determination of the concentrations is effected by means of mass spectrometry. Disclosed is the use of μ-opioid receptors as target molecules, morphine as a marker and naloxone as a ligand in different concentration.

Dollinger et al. U.S. Pat. No. 5,891,742, “Affinity selection of ligands by mass spectroscopy,” discloses a method in which compounds are selected from a combinatorial library by contacting the library with a target (human urokinase plasminogen activator), separating non-binding compounds from compound-target complexes, and analyzing the complexes or eluted compound by mass spectroscopy.

Neiens et al., “Simultaneous Multiple MS Binding Assays for the Dopamine, Norepinephrine, and Serotonin Transporters,” ChemMedChem 13(5) 453-463 (2018), discloses label-free, mass-spectrometry-based binding assays (MS Binding Assays), targeting monamine transporters. Human dopamine, norepinephrine, and serotonin transporters (hDAT, hNET, and hSERT) are used in simultaneous binding experiments.

Grimm et al., “Development and validation of an LC-ESI-MS/MS method for the triple reuptake inhibitor indatraline enabling its quantification in MS Binding Assays,” Anal Bioanal Chem. 2015 January; 407(2):471-85 discloses an LC-MS/MS quantification method for indatraline, a highly potent nonselective inhibitor of the three monoamine transporters (for dopamine, DAT; for norepinephrine, NET; for serotonin, SERT), and its application to MS Binding Assays.

de Jong et al., “Development of a multiplex non-radioactive receptor assay: the benzodiazepine receptor, the serotonin transporter and the β-adrenergic receptor,” Rapid Comm. Mass Spectrom. 21:567-572 (2007), discloses a method in which a pool of receptors from rat cortical tissue, i.e. homogenized cortex, was combined with flunitrazepam (which binds to benzodiazepine binding sites [receptors]), MADAM (2-[2-[(dimethylamino)methyl]phenyl]sulfanyl-5-methylaniline;dihydrochloride, which binds to the serotonin transporter), and pindolol (beta blocker [adrenergic beta-antagonists]). Each ligand was incubated with its known displacer.

Bowes et al., “Reducing safety-related drug attrition: the uses of in vitro pharmacological profiling;” Nat. Rev. Drug Discov. 2012 December; 11(12):909-22 discloses the rationale for in vitro pharmacological profiling used at four major pharmaceutical companies. Proposed targets include GPCRs, ion channels, enzymes, neurotransmitter transporters, nuclear receptors.

### SUMMARY OF THE INVENTION

The following brief summary is not intended to include all features and aspects of the present invention, nor does it imply that the invention must include all features and aspects discussed in this summary.

The present invention, in various embodiments, is a multiplexed method for quantitating binding of a test compound to a target molecule and binding to off-target target molecules, comprising the steps of: (a) obtaining a mixture comprising target molecules from at least one of (i) a healthy or a non-healthy human or non-human tissue, and (ii) a synthetic protein preparation; (b) incubating said target molecules in a plurality of mixtures comprising ligands and test compounds, wherein said target molecules are incubated with different ligands; (c) after incubating, removing unbound ligands from said plurality of mixtures; then (d) isolating ligands that were bound to target molecules in said mixture of target molecules, ligands, and test compounds; (e) determining a quantity of ligand that was bound by a target molecule, by measuring ligands that were obtained in step (d), using mass spectrometry and a calibration curve; and (f) determining an affinity of the test compound for a target molecule in said mixture of target molecules using data obtained in step (e); and (g) measuring binding of said test compound to a predetermined target molecule and comparing said binding to binding of said test compound to off-target molecules.

In various embodiments, the present invention discloses a multiplexed method for quantitating binding of a test compound to a predetermined target molecule and also to binding to off-target target molecules, comprising the steps of: (a) obtaining a mixture comprising target molecules from at least one of (i) healthy or non-healthy human or non-human tissue, and (ii) a synthetic protein preparation; (b) incubating said target molecules in a plurality of mixtures comprising ligands and test compounds, wherein said target molecules are incubated with different ligands; (c) removing unbound ligands from said plurality of mixtures; then (d) isolating ligands that were bound to target molecules in said mixture of target molecules; (e) determining a quantity of ligand that was bound by target molecules, by measuring ligands that were obtained in step (d), using mass spectrometry and a calibration curve; (f) determining an affinity of the test compound for target molecules in said mixture of target molecules using data obtained in step (e); and (g) measuring binding of said test compound to a predetermined target molecule and comparing said binding to binding of said test compound to off-target molecules.

The multiplexing in the present methods can comprise multiple target molecules in the same mixture, wherein the target molecules do not exist in a single preparation in nature. In various embodiments, the present invention comprises a heterologous mixture of target molecules. In certain other embodiments, the present invention comprises a mixture of target molecules comprising at least one human target molecule or more than one human target molecule.

The present invention, in certain aspects, comprises methods as described above, wherein step (a) comprises obtaining the target molecule or target molecules from a crude extract. The present invention, in certain aspects, comprises a method as described above, wherein said step of obtaining target molecules comprises obtaining human target molecules. The extract may be present on ex vivo membranes of cerebral cortex, cerebellum, ventricular and hepatic membrane preparations.

In various embodiments, binding of a test compound to a predetermined target molecule may be any one of cerebral cortex, cerebellum, ventricular and hepatic membrane preparations. For example, a test compound is of interest for binding to the A1 receptor. Stimulation of the A1 receptor has a myocardial depressant effect by decreasing the conduction of electrical impulses. This makes adenosine a useful medication for treating and diagnosing excessively fast heart rates.

In various embodiments, binding of a test compound to the other target molecules may be considered off-target binding.

The present invention, in certain embodiments, comprises methods as described above, wherein step (c) comprises removing unbound ligands from the mixtures or plurality of mixtures using a glass filter. The present invention, in certain embodiments, comprises a method as described above, wherein step (d) comprises eluting the bound ligand from the glass filter using a solvent, then concentrating samples from the filter.

The present invention, in certain aspects, comprises methods as described above, wherein said mass spectroscopy comprises using liquid chromatography/electrospray ionization tandem mass spectroscopy. The present invention, in certain aspects, comprises a method as described above further comprising the step of determining a Kₒₙ and K_{off} of a test compound to the target molecule.

The present invention, in certain aspects, comprises methods as described above wherein said target molecules are present in a mixture of receptor target molecules that does not exist in nature. The present invention, in certain aspects, comprises a method as described above, wherein said target molecules are selected from the group consisting of Na channel, alpha1 beta-adenoreceptor, alpha 2 beta-adrenoceptor A1 (adenosine receptor), M1 (muscarinic receptor), 5-HT_{2A} (serotonin receptor), Alpha 1ns (adrenergic receptor), Alpha 2 ns (adrenergicD1 (dopamine receptor), and 5HTtrans (serotonin receptor).

In certain embodiments, the present invention discloses a method as described above comprising the step of determining a Kₒₙ and K_{off} of the test compound to the target molecule wherein K_{off} is determined by a displacement method or a dilution method.

The present invention, in certain embodiments, comprises a method as described above, wherein the ligands used to study target molecules may be selected from the group consisting CPX, pirenzepine, prazosine, RX821002, SCH233900, 8-OH-DPAT, EMD281014, paroxetine, D600, MK801, and naloxone.

The present invention, in various embodiments, comprises a multiplexed method for quantitating binding of at least two different test compounds (test compound C1, C2, et seq.) to at least two different receptor target molecules (receptor target RT1 for C1, RT2 for C2 et seq.), based on competitive binding between the test compounds and known binders for RT1 and RT2 (known binder B1, B2 et seq.), comprising: (a) providing a mixture comprising (i) test compounds C1 and C2; (ii) known binders B1, B2, and (iii) receptor target molecules RT1, RT2; (b) allowing complexes to form in said mixture between the test compounds C1, C2 et seq., RT1 and RT2, as well as B1 and B2; (c) separating compounds which do not form complexes with RT1, RT2 et seq. from said complexes; (d) isolating binders B1, B2 et seq. from complexes obtained in step (c) and passing isolated binders through a mass spectrometer to measure binding of test compounds C1 and C2 using mass spectroscopy; and (e) determining the relative affinities of C1 and C2 for RT1 and RT2, respectively.

For further clarification, the statement “et seq.” refers to a series of members of the series of materials that can be represented as Cₙ, Bₙ, and RTₙ, wherein n is between 2 and 40 or between 1 and 40 or between 2 and 50. This indicates, for example, that if n=10 there are 10 C, 10 B and 10 RT's.

For the sake of clarification, it is contemplated that the set of receptor target molecules (RT) test compounds (C), and known binders (B) contain between two and about 20 members (or more) in a single multiplex reaction.

The present invention, in various embodiments, comprises a method as described above, wherein the receptor target molecules RT1-RTₙ are in the mixture not found in nature in a single mixture or n the same tissue.

In many embodiments, the present invention discloses a multiplexed method for quantitating binding affinity of at least two different test compounds (test compound C1-Cₙ) to at least two different receptor target molecules (receptor RT1 for C1, RTₙ for Cₙ), based on competitive binding between the test compounds and known binders for RT1 and RT2 (known binder B1-Bₙ), comprising: (a) providing a mixture comprising (i) test compounds C1-Cₙ; (ii) known binders B1-Bₙ and (iii) receptor target molecules RT1-RTₙ; (b) allowing complexes to form in said mixture between the test compounds C1-Cₙ, RT1-RTₙ, and B1-Bₙ, (c) separating compounds which do not form complexes with their target molecules from said complexes; (d) isolating known binders from complexes obtained in step (c) and passing isolated binders through a mass spectrometer to measure binding of test compounds using mass spectroscopy; and (e) determining the relative affinities of compounds C1-Cₙ for RT1-RTₙ, respectively, wherein Cₙ, Bₙ, and RTₙ represent a series of members wherein n is between 2 and 40.

The present invention, in various embodiments, comprises a method as described above, wherein step (a) comprises obtaining receptor target molecules from a crude extract. The present invention, in certain aspects, comprises a method as described above, wherein said step of providing receptor target molecules RT1-RTₙ comprises providing human receptor target molecules. The present invention, in certain aspects, comprises a method as described above, wherein step (c) comprises separating using a glass filter and washing. The present invention, in certain aspects, comprises a method as described above, wherein step (d) comprises eluting the bound ligand from the filter using a solvent, then concentrating samples from the filter.

The present invention, in various embodiments, comprises a method as described above, wherein said mass spectroscopy comprises using liquid chromatography/electrospray ionization tandem mass spectroscopy. The present invention, in various other embodiments, comprises a method as described above, further comprising the step of determining a Kₒₙ and K_{off} of a test compound to the target molecule.

Further, the present invention discloses a multiplexed method for quantitating binding affinity of a test compound to a target molecule, comprising the steps of: (a) obtaining at least three target molecules as set forth in the chart below (Table 1); (b) incubating said target molecules in a plurality of mixture comprising ligands and test molecules; (c) removing unbound ligands from the mixtures; (d) isolating ligands that were bound to the target molecules; (e) determining the quantity of each ligand that was present on the target molecules by measuring ligands that were obtained in step (d) by mass spectrometry, using a calibration curve prepared with known concentrations of ligand; and (f) calculating an affinity of the test compound for the target molecule from the data obtained in step (e). The method as disclosed, wherein the same test compound is used with each target molecule. The method further comprises using target molecules with the ligands as shown in Table 2.

TABLE 1
Target molecule
Adenosine receptor A1
Muscarinic
acetylcholine receptor
5-HT_{2A} (serotonin)
Alpha-1A adrenergic
receptor
Alpha-2A adrenergic
receptor
Dopamine receptor D1
5HT transporter
5HT1a receptor
5HT2a receptor
Cave Ca channel
PCP receptor
Opioid receptor

TABLE 2

| Target molecule | Ligand |
|---|---|
| Adenosine receptor A1 | CPX |
| Muscarinic | Pirenzepine |
| acetylcholine receptor | |
| 5-HT_{2A} (serotonin) | EMD281014 |
| Alpha-1A adrenergic | Prazosine |
| receptor | |
| Alpha-2A adrenergic | RX82102 |
| receptor | |
| Dopamine receptor D1 | SCH23390 |
| 5HT transporter | paroxetine |
| 5HT1a receptor | 8-OH-DPAT |
| 5HT2a receptor | EMD281014 |
| Cave Ca channel | D600 |
| PCP receptor | MK801 |
| Opioid receptor | naloxone |

The present invention, in certain aspects, comprises a method as described above using the following combinations of receptor target molecules and ligands (Table 3):

TABLE 3

| Receptor target molecule | Ligand |
|---|---|
| Adenosine receptor A1 | CPX |
| Muscarinic acetylcholine | pirenzepine |
| receptor M1 | |
| 5HT1a | 8-OH-DPAT/5 |
| 5-HT_{2A} (serotonin) | EMD281014 |
| Alpha-1A adrenergic | Prazosine |
| receptor | |
| Alpha-2A adrenergic | RX82102 |
| receptor | |
| Dopamine receptor D1 | SCH23390 |
| 5HT transporter | paroxetine |
| Ca++ channel (“Cave”) | D600 |
| Mu opioid receptor | Naloxone |
| Sigma receptor/PCP | MK801 |
| receptor | |

The above receptor target molecules may be assayed with other ligands not listed in the above Table 3 or other receptor target molecules not listed in the above Table 3 may be assayed with ligands shown above.

In various embodiments, the present methods comprise a multiplex method for determining a Kₒₙ and K_{off} values of a test compound to a target molecule, comprising the steps of: (a) obtaining a mixture of target molecules from at least one of (i) healthy or non-healthy human or non-human tissue, and (ii) a synthetic protein preparation; (b) incubating said target molecules in a plurality of mixtures comprising ligands and test compounds, wherein said target molecules bind to different ligands and are incubated with different target molecules; (c) after incubating, removing unbound ligands from the mixtures; (d) isolating bound ligands that were bound to the target molecules; (e) determining a quantity of ligand that was bound by target molecules, by measuring ligands that were obtained in step (d) at defined time points in the reaction, using mass spectrometry and a calibration curve; and (f) calculating Kₒₙ or K_{off} of the test compound to a target molecule using data obtained in step (e).

In various embodiments, the present methods comprise a method wherein a Kₒₙ and K_{off} are determined in mixtures of different ex vivo membranes comprised of at least two of rat cortex, cerebellum, ventricular and hepatic membrane preparations. In certain aspects, the present methods comprise a method wherein membrane mixtures comprise at least two of receptor A1, A2A (h), A3 (h), M1, M2 (h), Alpha1ns, Alpha2 ns, D1, D2S (h), 5HT1a, 5HT2a, 5HTtrans, Cave, PCP, Opioid ns, AT2 (h), B2 (h), CB1 (h), CCK1 (CCKA), H4 (h), and CysLT1 (LTD4) (h). In certain aspects, the present methods comprise a membrane mixture comprising all of the listed receptors. In certain other embodiments, the present methods comprise a method wherein K_{off} is determined by a displacement method. In certain aspects, the present methods comprise a method wherein K_{off} is determined by a dilution method. In various embodiments, (h) stands for human.

In various embodiments, target molecules are receptors.

As described below, the same test compound may be used with the above different target receptor molecules and different ligands, generating information on target and off-target binding by the test compound.

Other features will be apparent from the accompanying figures and from the detailed description that follows.

### BRIEF DESCRIPTION OF THE DRAWINGS

Example embodiments are illustrated by way of example and no limitation in the tables and in the accompanying figures, like references indicate similar elements and in which:

FIGS. 1A and 1B shows an exemplary work flow for MS binding assays used to characterize binding of various ligands (target molecules, or receptor target molecules) to a test compound.

FIGS. 2A-2C shows correlation between radioligand binding and the present MS binding method in rat cortex sodium channels. FIG. 2A is a graph showing radioligand binding assay results of sodium channels. FIG. 2B is a graph showing MS binding assay results of veratridine. FIG. 2C is a graph showing MS binding assay results of batrachotoxine.

FIG. 3A is a graph showing concentration effect of WB4101 in the presence of Prazosine and RX821002. FIG. 3B is a graph showing concentration effect of Yohimbine in the presence of RX821002 and Prazosine.

FIGS. 4A-K shows a series of graphs showing results from a simultaneous binding assay employing rat cortex target molecules. FIG. 4A is a graph showing concentration effect of NECA in the presence of CPX at 5nM on rat cortex. FIG. 4B is a graph showing concentration effect of ATROPINE in the presence of Pirenzepine at 1 nM on rat cortex. FIG. 4C is a graph showing concentration effect of SEROTONINE in the presence of 8-OH-DPAT at 1 nM on rat cortex. FIG. 4D is a graph showing concentration of W94101 in the presence of Prazosine at 1 nM on rat cortex. FIG. 4E is a graph showing concentration effect of Yohimbine in the presence of RX821002 at 1 nM on rat cortex. FIG. 4F is a graph showing concentration effect of BUTACLAMOL in the presence of SCH23390 at 1 nM on rat cortex. FIG. 4G is a graph showing concentration effect of Zimelidine in the presence of Paroxetine at 1 nM on rat cortex. FIG. 4H is a graph showing concentration effect of SEROTONINE in the presence of EMD281014 at 1 nM on rat cortex. FIG. 4I is a graph showing concentration effect of D888 in the presence of D600 at 5 nM on rat cortex. FIG. 4J is a graph showing concentration effect of DAMGO in the presence of NALOXONE at 1 nM on rat cortex. FIG. 4K is a graph showing concentration effect of SKF10047 in the presence of MK801 at 5 nM on rat cortex.

FIG. 5 is a schematic workflow for using MS to determine binding kinetics of a test compound to its cognate receptor molecule.

FIG. 6A is a graph showing results of MS assay to determine association kinetics curve of CGP54626 on GABAB1_{b/2}. FIG. 6B is a graph showing results of MS assay to determine dissociation kinetics curve of GABAB1_{b/2} from CGP542626 at concentration of 1 nM by the displacement approach via the addition of 10 μM CPG52432. FIG. 6C is a graph showing results of MS assay to determine dissociation kinetics curve of GABAB1_{b/2} from CGP54626 at a concentration of 5 nM by the dilution approach.

### DETAILED DESCRIPTION

### Overview

Described here is a method of measuring a binding activity of a test compound to a receptor target molecule using a mixture of biologically relevant target molecule. Further described here are methods for measuring the binding activity of test compounds to various receptor (target) molecules using a heterologous mixture of biologically relevant target molecules. The target molecules in this assay may be used to assess off-target binding. In one aspect, the method uses a competitive binding assay using a target molecule or tissue that is known to bind to a ligand. As is known from principles of radioimmunoassays (RIA), dilution curves are constructed using various concentrations of the known ligand (or “marker”) and its binding to the target molecule. Unlike RIA, the markers in the present method need not be labeled or otherwise chemically modified. Binding of the test compound, with ligand, and the tissue (target molecules) are then measured at a known concentration; then, the MS signal is compared to the MS signals obtained in the dilution curve. The effectiveness of the test compound in binding to the target molecule is then known, and an IC₅₀ or EC₅₀ can be determined.

In the present methods, binding characteristics of test compounds to different target molecules can be determined in a multiplex procedure. The present methods also relate to in vitro methods for studying drug candidates.

The present methods can use commercially available high performance liquid chromatography (HPLC) and MS equipment. The MS format can be electrospray from a well, or use a matrix in a matrix-assisted laser desorption/ionization (MALDI) format, or use other ionization technique.

The present methods can be automated using laboratory robotics. All the separations and reactions in the method are contained in the same sample well until such time as recovered molecules are input into the HPLC. A sample plate with any number of desired wells can be used.

A variety of target molecules may be prepared for use in the present methods. Crude or purified extracts may be used, e.g. by methods disclosed in U.S. Pat. No. 4,446,122, “Purified human prostate antigen;” U.S. Pat. No. 6,548,019, “Device and methods for single step collection and assaying of biological fluids;” Magomedova et al., “Quantification of Oxysterol Nuclear Receptor Ligands by LC/MS/MS;” Methods Mol. Biol. 2019; 1951:1-14; and Wang, “Purification and autophosphorylation of insulin receptors from rat skeletal muscle,” Biochim Biophys Acta. 1986 Aug. 29;888(1):107-15, all hereby incorporated herein by reference.

Use of a glass filter to prepare a sample for MS analysis may be carried out a described, e.g., in Merck Millipore, “Perfection in preparation for better mass spectra,” Merck Millipore product sheet, 2012 retrieved at http (colon slash slash www. merckmillipore.com/INTERSHOP/web/WFS/Merck-JP-Site/ja_JP/-/JPY/ShowDocument-Pronet id=201306.10657.

### Definitions

Unless defined otherwise, all technical and scientific terms used herein have the same meaning as commonly understood by those of ordinary skill in the art to which this invention belongs. Although any methods and materials similar or equivalent to those described herein can be used in the practice or testing of the present invention, the preferred methods and materials are described. Generally, nomenclatures utilized in connection with, and techniques of, cell and molecular biology and chemistry are those well-known and commonly used in the art. Certain experimental techniques, not specifically defined, are generally performed according to conventional methods well known in the art and as described in various general and more specific references that are cited and discussed throughout the present specification. For purposes of the clarity, following terms are defined below.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods, cells, compositions and kits. The upper and lower limits of these smaller ranges may independently be included in the smaller ranges and are also encompassed within the methods, cells, compositions and kits, subject to any specifically excluded limit in the stated range. Where the stated range includes one or both of the limits, ranges excluding either or both of those included limits are also included in the methods, cells, compositions and kits.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods, cells, compositions and kits are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The term “affinity” is used in a conventional sense to refer binding affinity. Binding affinity is the strength of the binding interaction between a single biomolecule (e.g. protein) to its ligand/binding partner (e.g. drug or inhibitor). Binding affinity is typically measured and reported by the equilibrium dissociation constant (Kd), which is used to evaluate and rank order strengths of bimolecular interactions. Accordingly, binding kinetics describe how fast a compound binds to its target and how fast it dissociates from it. So, it measures two things—the on-rate and the off-rate. See, U.S. Pat. No. 5,324,633A, “Method and apparatus for measuring binding affinity.”

The term “ligand,” or “binder” is used herein to refer to a material that is known to bind to a given receptor or other target molecule. This term may be further understood by reference to Siimans et al., U.S. Pat. No. 5,814,498, “Methods of enumerating receptor molecules for specific binding partners on formed bodies and in solution,” hereby incorporated by reference as providing concepts of competitive binding.

A “mixture of targets” or target molecules means a mixture of structurally different targets or other receptor target molecules. As a non-limiting example, this mixture can comprises glutamate receptors, D1dopamine receptors, and nicotinic acetylcholine receptors. These receptors may be present in a single tissue type, such as a brain cerebral cortex of an animal or may not be present in a single tissue type. The mixture of targets can also include, for example, glutamate receptors (from cerebral cortex) and VEGF receptors (from endothelial cells). See below, “heterologous mixture of receptor target molecules”.

A “heterologous mixture of target molecules” refers to a mixture of different target molecules that are not found in nature in a single tissue, or, if present in the same tissue, have different biological functions. As a non-limiting example, this mixture may comprise more than one tissue selected from the group consisting of engineered cells expressing G-protein-coupled receptors (GPCRs), animal-sourced cerebral cortex (having 15 different targets molecules, as described e.g. in Zilles et al., “Multiple Transmitter Receptors in Regions and Layers of the Human Cerebral Cortex,” Front Neuroanat. 11:78 (2017)), cerebellum, cardiac, muscle (including cardiac ion channels), biological enzymes (e.g. COX2, COX1, MAO, PDE4, Ache, LCK), nuclear receptors (e.g. AR and NR3C1) and nucleic acid molecules.

The target molecules will comprise desired binding and binding that is not desired, known as off-target binding. As discussed above, off-target binding is generally avoided for safety reasons. See Bowes et al. and Eurofins Safety Panels, h-t-t-ps-:slash-slash www(dot).eurofinsdiscoveryservices.com/cms/cms-content/services/safety-and-efficacy/safety-pharmacology/safety-panels/, discloses a selection of in vitro Safety Panels.

The term “MS” means mass spectrometry. In the present method, a variety of mass spectrometry methods can be used, e.g., AMS (Accelerator Mass Spectrometry), Gas Chromatography-MS, Liquid Chromatography-MS, ICP-MS (Inductively Coupled Plasma-Mass spectrometry), IRMS (Isotope Ratio Mass Spectrometry), Ion Mobility Spectrometry-MS, MALDI-TOF, SELDI-TOF, Tandem MS, TIMS (Thermal Ionization-Mass Spectrometry), and SSMS (Spark Source Mass Spectrometry).

The term “multiplex” refers to an assay in which multiple different analyses are conducted in a single procedure, using different target molecules having different ligands. The process may also comprise having different test compounds. The binding of a test compound to different target molecules that do not exist together in nature can be carried out simultaneously in a multiplex assay. Furthermore, a multiplex assay may produce multiple results from a single mixture of target receptors and yield a binding profile to different target molecules that will elucidate off target binding and, thus, safety.

The term “liquid chromatography/electrospray ionization tandem mass spectroscopy” may be further understood by reference to, e.g., Bandu et al., “Liquid Chromatography Electrospray Ionization Tandem Mass Spectrometric (LC/ESI-MS/MS) Study for the Identification and Characterization of In Vivo Metabolites of Cisplatin in Rat Kidney Cancer Tissues: Online Hydrogen/Deuterium (H/D) Exchange Study,” PLosOne 2015 Aug. 5:10(8).

The term “receptor target molecule” or “target molecule” or “receptor molecule” refers to a biological compound for which binding of a test compound is to be measured. A given receptor target molecule may be present in a target tissue obtained from a cell, an animal (human or nonhuman). It may be produced by recombinant DNA, or otherwise synthesized so as to contain one or more target molecules of interest. It may be membrane bound or exist in a liquid mixture, such as an enzyme. Potential receptor target tissues used herein may be cerebral cortex, brain astrocytes, neuronal tissues (including neuronal stem cells), cardiac tissues, liver tissues, blood tissues, kidney tissues, eye tissues, gut tissues, etc. The target tissue may be normal or diseased. It may be derived from an animal source or a human source. The term “heterologous mixture of target molecules “refers to tissues or cell lines from different origins, as illustrated above. Tissues may be different tissues if from the same tissue, but the tissues have different structure, due to disease, state of development, or the like.

The term “synthetic protein preparation” means a preparation of a protein that was synthesized rather than obtained from a native cell or tissue. The synthetic protein preparation may be synthesized by recombinant DNA methods, peptide synthesis, or the like.

The term “test compound” means material that is under study for its binding affinity for target molecules. It will interact with and compete with the known ligand (marker) if it binds to a target molecule that is also bound by the marker. The test compound may be a potential drug, as well as metabolites of such drug. It may be a small molecule or a protein or polynucleotide. It may also be a molecule that is being tested because of its potential in vivo diagnostic application.

### Generalized Method and Apparatus

The present methods can be adapted to a wide variety of test compounds and a wide variety of targets for which binding characteristics of test compounds are to be elucidated. Of particular interest is the study of test compounds that are drug candidates for in vivo human use. The binding of test compounds to various target molecules represented by various tissues are studied in the present methods. Binding is either desired for a therapeutic effect or is not desired to avoid off target effects, as a matter of drug safety. As such, the present methods find use, e.g., in the identification of potential human therapeutics and their potential undesired binding to various human tissues expressing potential targets for test compound binding.

### EXAMPLES

### Example 1: Workflow

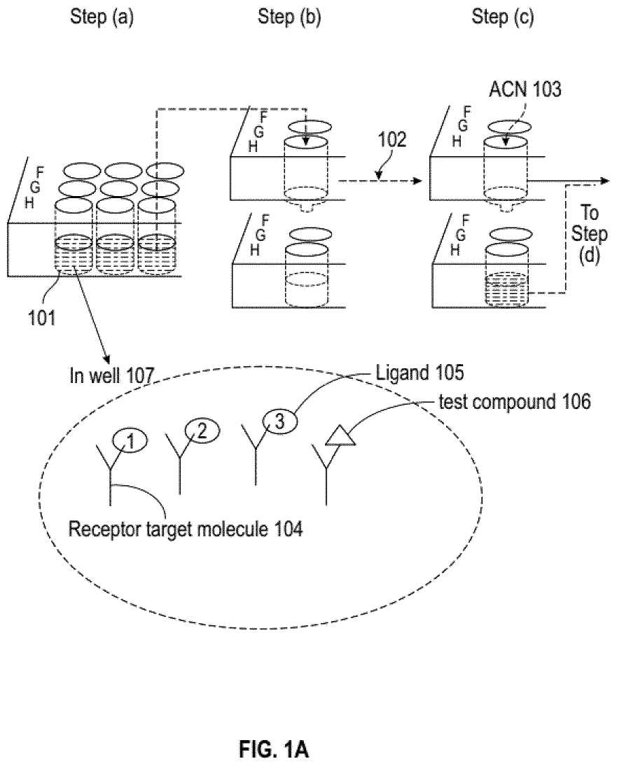

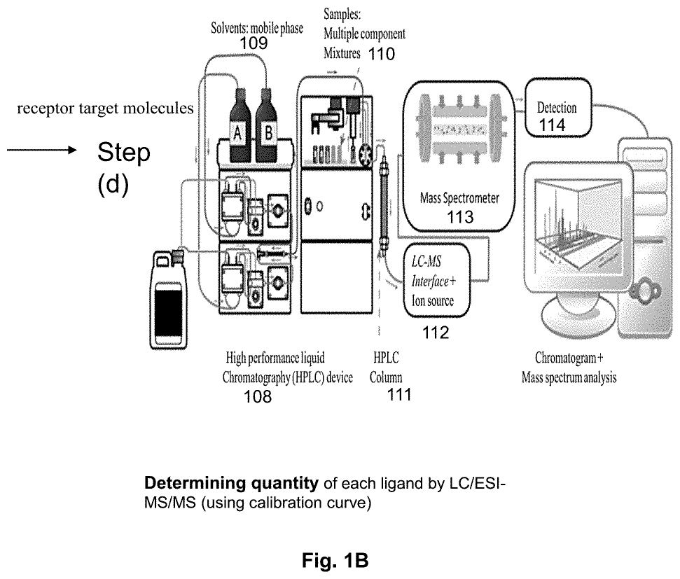

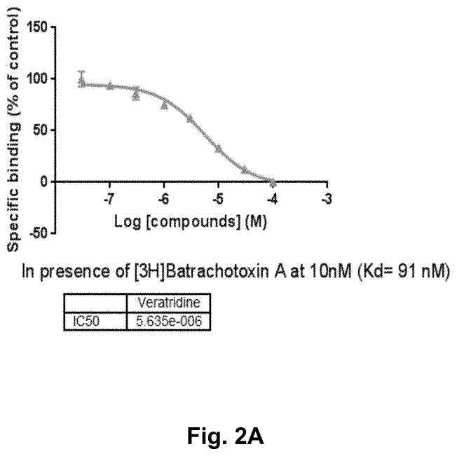

Referring now to FIGS. 1A and 1B, the present methods are shown to comprise a series of incubation, separation, and wash steps that lead to the direct or indirect quantitation of test compounds that were competed off a target molecule by a known binder ligand. See Insert in FIG. 1A illustrating one test well 107. The ligands are designated 1, 2, and 3 to designate different ligands 105 binding to different receptor target molecules 104 in a single well.

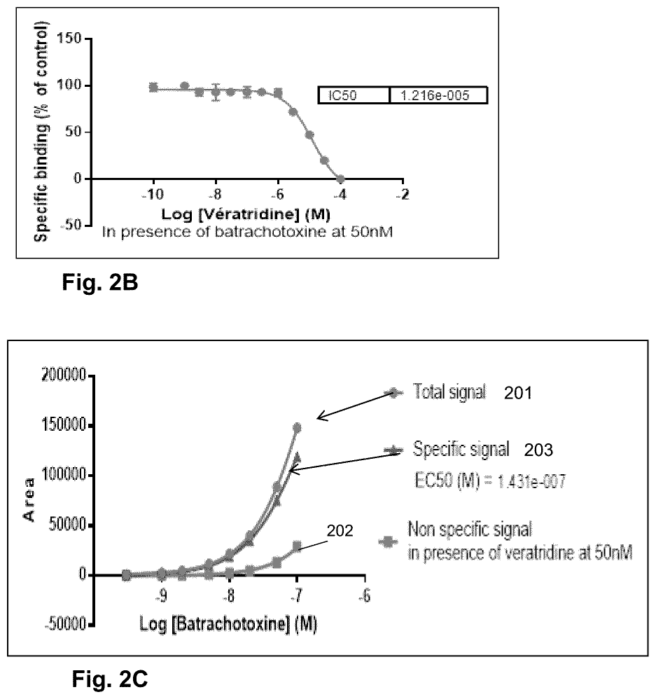

FIGS. 1A-1B show incubation of a heterologous mixture of receptor target molecules with ligands (known binders), and different test compounds. The wells, vials, or other containers contain target molecules. As shown in 101, step (a), a given well in a multi-well plate can contain mixtures of target molecules 104, ligands (known binders) 105, and different test compounds 106. As shown in the insert 107, the target molecules 104 may bind to different ligands 105, labeled as 1, 2, and 3. The differentiation and identification of the ligands is carried out by MS. Various wells contain different amounts of molecules, whereby the results from the analysis of the wells in FIGS. 1A and 1B can be used for the drawing of concentration curves, as shown in FIGS. 2-6. Next, as shown at FIG. 1A step (b), unbound ligands are separated from the complexes in the wells. Then, as shown at (c), ligands that were bound to the target molecules are separated from the mixture and removed from the well for use in FIG. 1B step (d). Removal of the bound ligands in step (c) can be facilitated by the use of acetonitrile 103 and a glass filter which allows passage only of unbound ligands. Various organic solvents can be used in this step, as well as other recovery steps for the preparation of ligands for use in step (d).

After recovery of previously bound ligand molecules, the amount of ligand obtained from each well is quantitated by liquid chromatography and electrospray MS (mass spectroscopy) (step (d) in FIG. 1B). An LC/ESI-MS/MS method is used, so that liquid chromatography will reduce the amount of irrelevant mass spectroscopy peaks when the mass spectrometer is used to identify and quantitate the various ligands.

FIG. 1B shows a HPLC device 108, solvents to produce a mobile phase 109, a unit for preparing component mixtures 110, and an HPLC column 111 that outputs to an ion source 112 and mass spectrometer 113. The exemplary chromatogram and mass spec analysis reveals the amount of binding of test compound to the target molecules 114.

In another embodiment of the present methods, a fixed amount of test compound may be measured under different concentrations of ligands (known binders). That is, an excess of test compound is used, if such is available and different amounts of ligands are used. Ligand is competed off the test compound-target molecule complex to determine binding behavior of the test compound to the target molecule.

Further, FIGS. 1A and 1B show a preparation of receptor target molecules is placed in test wells, vials or other containers. It may be a crude tissue extract containing the receptor target molecules. The tissue may be blood, serum, cerebral spinal fluid, brain segment (cerebral cortex, cerebellum, brain stem, etc.), extracts of glands (adrenal glands, pituitary glands, thymus, pancreas, ovary, thyroid, testicle, hypothalamus, etc.), or organ tissue such as cardiac, skeletal muscle, kidney, lung, etc. The tissue may be derived from human or non-human or animal tissue. It may be normal or diseased. The receptor target molecules need not be purified, and are selected based on the anticipated use of the test compound, the availability of known ligands, and the purpose of the assay. The purpose of the assay may be to obtain a safety profile, where a large variety of potential target molecules will be tested with the test compound to evaluate undesired binding.

In addition, the receptor target molecules may be prepared without the use of endogenous tissue, but, rather, prepared by rDNA or protein synthesis. Known cloned receptors useful in the present methods include H3 histamine receptors, opioid receptors, G protein-coupled receptors, vanilloid receptors, glutamate receptors, etc.

The multiplex methods here are carried out on multiple reaction areas (wells) shown as F, G and H, for an 8 row, 96 well plate (as shown in 101). 384 well plate or other multi-well formats can be used. In this example, receptor target molecule were prepared with ligand and test compounds and incubating the multiplex at 2 h, 37° C. in a 96 well plate. As shown in the insert below 107 panel (a), a well comprises a number of receptors bound to ligands 105 and a number of receptors 104 bound to the test compound 106 instead of the ligand 105.

After incubation in step (a), the complexes of target molecule receptors bound to target molecules are separated from unbound ligands and free target molecules by filtration. Vacuum filtration is simultaneously applied over the plate (FIG. 1A, step (b). Alternatively, step (b) may use wells that comprise a piston or syringe to separate the bound complexes from unbound molecules. Alternatively, the receptor target molecules may be tagged for separation from the wells. In this embodiment, unbound molecules can be easily removed.

Once the bound ligand is isolated from free ligands, the complexes can be washed with a low ionic strength buffer and finally eluted using an organic buffer or high ionic strength buffer, effectively isolating ligand-bound receptors for processing in step (c). The receptors may also be tagged with magnetic beads and processed as described above. Accordingly, as shown in FIG. 1A step (b) 102, the separated ligand-receptor complex is further treated so as to separate the ligand from the bound target receptors molecules, e.g. by elution by acetonitrile (FIG. 1A, step (c), 103). In step (d), (FIG. 1B), the isolated ligand molecules from step (c) are analyzed directly using LC electrospray MS-MS (liquid chromatography positive ion electrospray ionization tandem mass spectrometry).

Referring now to FIG. 1B, step (d), the ligand mixture is cleaned up by liquid chromatography and analyzed by mass spectroscopy. The image used was taken from Wikipedia “Liquid chromatography-mass spectrometry,” https(colon slash slash en.wikipedia(dot)org/wiki/Liquid chromatography-mass spectrometry, retrieved Jun. 28, 2019. As noted there, Mass spectrometry (MS) is an analytical technique that measures the mass-to-charge ratio (m/z) of charged particles (ions). Although there are many different kinds of mass spectrometers, all of them make use of electric or magnetic fields to manipulate the motion of ions produced from an analyte of interest and determine their m/z ratio. The basic components of a mass spectrometer are the ion source, the mass analyzer, the detector, and the data and vacuum systems. The ion source is where the components of a sample introduced in a MS system are ionized by means of electron beams, photon beams (UV lights), laser beams or corona discharge. In the case of electrospray ionization, the ion source moves ions that exist in liquid solution into the gas phase. The ion source converts and fragments the neutral sample molecules into gas-phase ions that are sent to the mass analyzer. While the mass analyzer applies the electric and magnetic fields to sort the ions by their masses, the detector measures and amplifies the ion current to calculate the abundances of each mass-resolved ion. In order to generate a mass spectrum that a human eye can easily recognize, the data system records, processes, stores, and displays data in a computer. In the example, electrospray ionization MS is used.

A calibration curve with known concentrations is used to quantify the amount of test compound that competed off the ligand and bound to the receptor test molecule. Other different mass spectroscopy methods, as detailed above can be used, provided that they do not produce excessive extraneous data.

It should be noted that the known binder, i.e. the marker, is unlabeled (as is the test compound). This is a key advantage of the present MS method over the RIA (radioimmunoassay) method. RIA is also based on competition between a known binder and a test compound, but requires that the marker be radio-labelled in order to achieve the desired sensitivity. In an alternative embodiment, a label such as deuterium can be added for increased sensitivity.

Further details on liquid chromatography/electrospray ionization tandem mass spectroscopy may be found in Becker, U.S. Pat. No. 6,835,927, “Mass spectrometric quantification of chemical mixture components,” hereby incorporated by reference.

Thus, FIGS. 1A and 1B shows a series of incubation and washing steps for the disclosed assay for a direct or indirect quantitation of test compounds wherein in step (a) a given well 101 in a multi-well plate comprises a mixture receptor target molecule 104, a ligand (known binder) 105, and a test compound 106. Further, as shown in the insert 107, the target molecule may bind to different ligands labelled as 1, 2, and 3. The mixture is allowed to incubate for 2 hr at 37° C. in multi-well plate. Following incubation in step (a), vacuum filtration is applied 102 in the multiple well plate for the separation of bound receptor target molecule from unbound ligands and free target molecules as shown in step (h). Step (c) shows the separated ligand receptor complex is further washed with a low iconic strength buffer such as by elution by acetonitrile 103 so as to separate the ligand from the bound target receptors molecule before moving to step (d) of the disclosed binding assay. In step (d), (FIG. 1B), the isolated ligand molecules from step (c) are analyzed directly using LC electrospray MS-MS (liquid chromatography positive ion electrospray ionization tandem mass spectrometry).

### Example 2: Comparability Between Present MS Method and RIA Method

Referring now to FIG. 2A, a radioligand binding assay of sodium (Na) channel and its comparison to the present MS method, shown in FIGS. 2B and 2C. FIG. 2B shows specific binding of veratridine in the presence of batrachotoxin at 50 nM. This experiment was done with sodium channels as the receptor target molecules. The ligand (known binder) may be considered to be batrachotoxin, which binds to and irreversibly opens the sodium channels of nerve cells and prevents them from closing. The test compound is the neurotoxin veratridine, which acts by binding to and preventing the inactivation of voltage-gated sodium ion channels in heart, nerve, and skeletal muscle cell membranes.

The SNR (signal to noise) was determined as follows (Table 4):

TABLE 4

| SNR | 6 | 8 | |
|---|---|---|---|
| Batrachotoxin | (Kd = 91 nM) | 143 nM | (EC50) |
| Veratridine | 5.6 μM (IC50) | 12.2 μM | (IC50) |

FIG. 2C shows an indication of the specificity of the binding. The line 201 in FIG. 2C indicates the total signal, the line 202 indicates the signal associated with the non-specific binding in the presence of veratridine and the line 203 indicates the specific signal. In this case, the binding of batrachotoxin was determined in membranes which were pre-incubated with a competitor (veratridine) known to bind to the same site. This is how one may determine if the specific ligand is not binding non-specifically to the filter, plastic or other sites.

Materials and methods:

### Rat Cortex Membrane Preparation

Rat cortexes from Wistar male rats were harvested and transferred to 50 mM Tris-HCl (pH, 7.4) and homogenized by a polyton. The homogenate was centrifuged 50 000 g for 15 minutes at 4° C. The resultant pellet was washed in lyses buffer containing 50 mM Tris-HCl (pH, 7.4) containing 1 μg/ml Leupeptin and 1 μM Pepstatin and was centrifuged 50 000 g for 15 minutes at 4° C. The pellet was finally resuspended in a smaller volume of lyses buffer and the final protein concentration was determined according to the Bradford method using bovine serum albumin as a standard.

### Filtration and Elution of Samples

Incubation was terminated by filtration after transfer of the binding sample (aliquot of 200 μl per well) onto 96-well glass filter plates and subsequently filtered rapidly under vacuum the membrane fraction bound to the filters were rinsed several times with wash buffer (50 mM Tris-HCl and 150 mM NaCl) on a vacuum manifold. Membrane filters were pretreated for 1 hour with 50 mM Tris-HCl and 0.3% of Polyethyleneimine solution (PEI).

The filters were dried for one hour at 50° C. and cooled to room temperature before elution of Batrachotoxin using a acetonitrile (contained 100 pM of antipyrine as an internal standard) via a vacuum manifold. Relative quantification of ligand in each sample was performed by UHPLC-MS-MS, the ratio area of ligand and internal standard was used.

### UHPLC-MS/MS Method Development

UHPLC-QQQ analysis was performed by a 1290 Infinity Binary LC system (Agnelli Technologies, Waldbronn, Germany) coupled to a Q-TRAP 5500 mass spectrometer with an ESI Turbo V ion source (SCIEX, Foster City, Calif., USA).

Chromatographic separation was performed on C₁₈ column (Poroshell 120 EC-C18, Agilent). The injection volume was 20 μl (full loop injection). The mobile phase consisted of two solutions including solvent A (0.1% formic acid and 6 mM ammonium acetate in water) and solvent B (0.1% formic acid and 6 mM ammonium acetate in acetonitrile), the column was thermostated in an oven at 35° C. and the flow rate was 650 μl/min.

The chromatographic gradient used for C18 column; initial composition of B was 0% during 0.3 min and increased to 80% from 0.3 to 0.9 min then 100% was reached at 1 min until 1.3 min, followed by re-equilibration to initial condition during 0.3 min.

For MS analysis, data were acquired using electrospray ionization (ESI) in positive mode, the Ion Spray Voltage was set at 5 500 V. The desolvation in source was accomplished using the following set parameters: Temperature (TEM) at 600° C., Ion Source Gas 1 (GS1) at 40 PSI, Ion Source Gas 2 (GS2) at 50 PSI, and Curtain Gas (CUR) at 50 PSI. The specific parameters of MRM method which to permit to quantify and monitored the ligand (Batrachotoxin) is described in Table 5. Raw Data were processed in Sciex Analyst and individual AUC (area under the curve) for each analyte in each sample was determined using the MultiQuant software.

TABLE 5
MRM method

| Q1 Mass | Q3 Mass | Time | | DP | EP | CE | CXP |
|---|---|---|---|---|---|---|---|
| (Da) | (Da) | (msec) | ID | (volts) | (volts) | (volts) | (volts) |
| 539.2 | 400.2 | 150 | Batrachotoxin | 140 | 10 | 23 | 12 |

DP: declustering potential, EP: entrance potential, CE: collision energy and CXP: Collision Cell Exit Potential.

### Binding by MS Experiments

### Optimal Concentration of Ligand Determination

Cortex membrane preparations containing the sodium channel (Na⁺) site 2 receptor and Batrachotoxin were incubated in triplicate in assay buffer (50 mM Hepes/Tris-HCl, 0.8 mM MgSO₄, 5 mM KCl, 7.5 mg/l scorpion venon, 2 mM MgCl2, 10 μg/ml trypsin, 1 g/l glucose, 130 mM chloline, 1 μg/ml leupeptin, 1 μg/ml pepstatin and 0.1% BSA) in polypropylene 96-deep-well plates at 37° C. Initially, 12 concentrations (in range from 10 μM to 300 nM) of Batrachotoxin was co-incubated for 60 minutes at 37° C., with 1 concentration (200 μg/well) of the rat cortex membrane preparation.

Non-specific binding was determined by the co-incubation with 10 μM verapamil.

The incubation was terminated by filtration after transfer of the total volume of the binding reaction to a filter plate. The remaining quantity of Batrachotoxin was determined by UHPLC-MS/MS.

### For Saturation Assays:

Membrane aliquots containing 200 μg of rat cortex membrane preparation were incubated in triplicate in the presence of 50 nM of Batrachotoxin in a total volume of 200 μl of assay buffer. Incubation was terminated by filtration after incubation for 60 minutes at 37° C.

Non-specific binding was determined by the co-incubation with 10 μM of verapamil.

The incubation was terminated by filtration after transfer of the total volume of the binding reaction to the filter plate. The remaining quantity of Batrachotoxin was determined by UHPLC-MS/MS.

### Mass Binding Competitive Assays:

The ligand displacement assays were performed using eight concentrations of the competing ligand, Veratridine (in a range from 0.1 nM to 100 μM) in triplicate. Incubation was terminated by filtration after incubation for 60 minutes at 37° C. The remaining quantity of Batrachotoxin was determined by UHPLC-MS/MS.

### Example 3: Multiplexing with 2 Simultaneous Targets

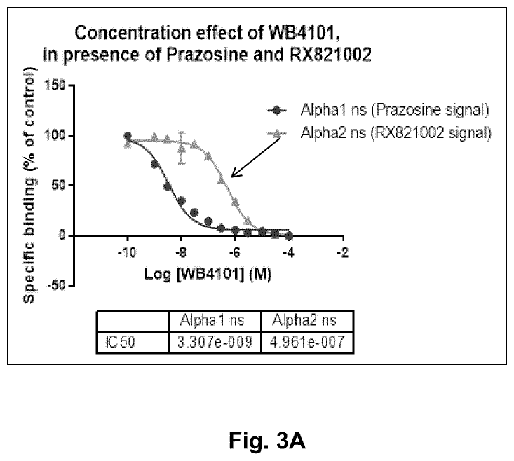

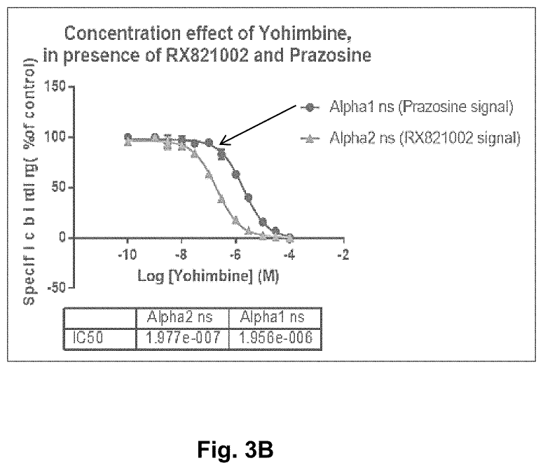

FIG. 3A is a graph showing a simultaneous binding experiment with alpha1 and alpha 2 beta-adrenoceptors. The target molecules are comprised in rat cortex, which contains both alpha 1 and alpha 2 beta adrenoceptors. The test compound is WB4101 and the ligands are prazosine and RX821002. FIG. 3B shows a simultaneous binding determination with alpha1 and alpha 2 beta-adrenoceptors using yohimbine as a test compound and the same target molecules and ligands as in FIG. 3A.

Now referring to FIG. 3A in detail. A rat cortex preparation was used to measure the effect of compounds on two different target molecules, in this case α1B-adrenergic receptor and the α2B-adrenergic receptor. The two receptors are structurally and functionally different. The human alpha-1A adrenergic receptor (ADRA1A) has a canonical length of 466 amino acids and a mass of 51,487 da. The human alpha-2A adrenergic receptor (ADRA2A) has a canonical length of 450 amino acids and a mass of 48,957 da.

WB4101 is a known antagonist of the α1B-adrenergic receptor. Prazosine is a drug known as a binder of the alpha-1 (α1) adrenergic receptor, which is a G protein-coupled receptor (GPCR). These receptors are found on vascular smooth muscle. RX821002 is a potent, selective α2-adrenoceptor antagonist.

This example used target molecule comprising both alpha 1 and alpha 2 beta adeno receptors incubated with WB4101 (test compound) in the presence prazosine (ligand, or “marker” for alpha 1) and RX821002 (ligand, or “marker” for alpha 2) as shown in FIG. 3A. In FIG. 3B, yohimbine (test compound) was tested in the presence of in the presence prazosine (marker for alpha 1) and RX821002 (marker for alpha 2). The signals are indicated ns for non-specific.

As shown in FIG. 3A, both prazosine and RX821002 were shown specifically to bind to alpha 1 and alpha 2 adrenergic receptor (respectively). FIG. 3B shows a simultaneous binding determination with alpha1 and alpha 2 using yohimbine as a test compound and the same targets and ligands as in FIG. 3A.

### Rat Cortex Membrane Preparation

Rat cortexes from Wistar male rats were harvested and transferred to 50 mM Tris-HCl (pH, 7.4) and homogenized by a polyton. The homogenate was centrifuged 50 000 g for 15 minutes at 4° C. The resultant pellet was washed in lyses buffer containing 50 mM Tris-HCl (pH, 7.4) containing 1 μg/ml Leupeptin and 1 μM Pepstatin and was centrifuged 50 000 g for 15 minutes at 4° C. The pellet was finally resuspended in a smaller volume of lyses buffer and the final protein concentration was determined according to the Bradford method using bovine serum albumin as a standard.

### Filtration and Elution of Samples

Incubation was terminated by filtration after transfer of the binding sample (aliquot of 200 μl per well) onto 96-well glass filter plates and subsequently filtered rapidly under vacuum the membrane fraction bound to the filters were rinsed several times with wash buffer (50 mM Tris-HCl and 150 mM NaCl) on a vacuum manifold. Membrane filters were pretreated for 1 hour with 50 mM Tris/HCl and 0.3% of Polyethyleneimine solution (PEI).

The filters were dried for one hour at 50° C. and cooled to room temperature before elution of ligands using a acetonitrile (contained 100 μM of antipyrine as an internal standard) via a vacuum manifold. Relative quantification of ligand in each sample was performed by UHPLC-MS-MS, the ratio area of ligand and internal standard was used.

### UHPLC-MS/MS Method Development

UHPLC-QQQ analysis was performed by a 1290 Infinity Binary LC system (Agilent Technologies, Waldbronn, Germany) coupled to a Q-TRAP 5500 mass spectrometer with an ESI Turbo V ion source (SCIEX, Foster City, Calif., USA).

Chromatographic separation was performed on C₁₈ column (Poroshell 120 EC-C18, Agilent). The injection volume was 20 μl (full loop injection). The mobile phase consisted of two solutions including solvent A (0.1% formic acid and 6 mM ammonium acetate in water) and solvent B (0.1% formic acid and 6 mM ammonium acetate in acetonitrile), the column was thermostated in an oven at 35° C. and the flow rate was 650 μl/min.

The chromatographic gradient used for C18 column; initial composition of B was 0% during 0.3 min and increased to 80% from 0.3 to 0.9 min then 100% was reached at 1 min until 1.3 min, followed by re-equilibration to initial condition during 0.3 min.

For MS analysis, data were acquired using electrospray ionization (ESI) in positive mode, the Ion Spray Voltage was set at 5 500 V. The desolvation in source was accomplished using the following set parameters: Temperature (TEM) at 600° C., Ion Source Gas 1 (GS1) at 40 PSI, Ion Source Gas 2 (GS2) at 50 PSI, and Curtain Gas (CUR) at 50 PSI. The specific parameters of MRM method which to permit to quantify and monitored the prazosine (ligand, or “marker” for alpha 1) and RX821002 (ligand, or “marker” for alpha 2) are described in Table 6. Raw Data were processed in Sciex Analyst and individual AUC (area under the curve) for each analyte in each sample was determined using MultiQuant software.

TABLE 6

| Q1 Mass | Q3 Mass | Time | | DP | EP | CE | CXP |
|---|---|---|---|---|---|---|---|
| (Da) | (Da) | (msec) | ID | (volts) | (volts) | (volts) | (volts) |
| 384.300 | 231.162 | 100 | Prazosine | 140 | 10 | 56 | 9 |
| 235.100 | 203.000 | 100 | RX821002 | 40 | 10 | 21 | 23 |

DP: declustering potential, EP: entrance potential, CE: collision energy and CXP: Collision Cell Exit Potential.

### Binding by MS Experiments

### Optimal Concentration of Ligand Determination

Rat cortex membrane preparations containing both alpha 1 non-selective (α1 NS) and alpha 2 non-selective (α2 NS) receptors were co-incubated with and Prazosine (specific ligand of α1 NS) and RX821002 (specific ligand of α2 NS) simultaneously. The assay was performed in triplicate in the assay buffer (50 mM Tris-HCl, 5 mM EDTA/Tris, 150 mM NaCl, 5 mM KCl, 2 mM MgCl2 and 0.1% BSA) in polypropylene 96-deep-well plates at 22° C. Initially, 12 concentrations (in a range from 0.1 nM to 300 nM) of Prazosine and RX821002 were co-incubated for 60 minutes at 22° C., with 3 concentrations (200 μg/well) of the rat cortex membrane preparations.

Non-specific binding was determined by the co-incubation with 10 μM WB 4101 and Yohimbine.

The incubation was terminated by filtration after transfer of the total volume of the binding reaction to a filter plate. The remaining quantity of both Prazosine and RX821002 was determined by UHPLC-MS/MS.

### Mass Binding Competitive Assays:

The ligand displacement assays was performed using 12 concentrations of the competing ligands, WB4101 (inhibitor of α1 NS) and Yohimbine (inhibitor of α2 NS) (in a range from 0.1 nM to 100 μM), and 0.3 nM of Prazosine and 1 nM of RX821002. They were co-incubated with 200 μg/well of rat membrane cortex in assay buffer, in triplicate. Incubation was terminated by filtration after incubation for 60 minutes at 22° C. The remaining quantity of both Prazosine and RX821002 was determined by UHPLC-MS/MS to be an alpha-2 adrenergic antagonist.

### Example 4: Multiplexing Different Target Molecules

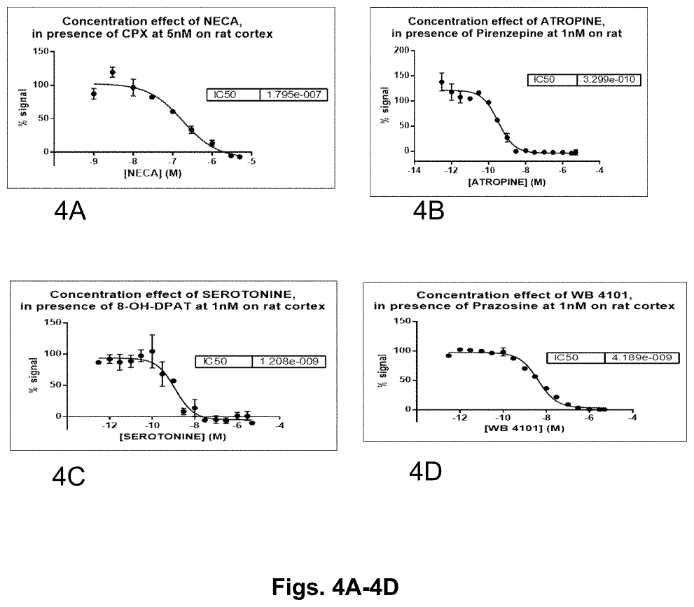

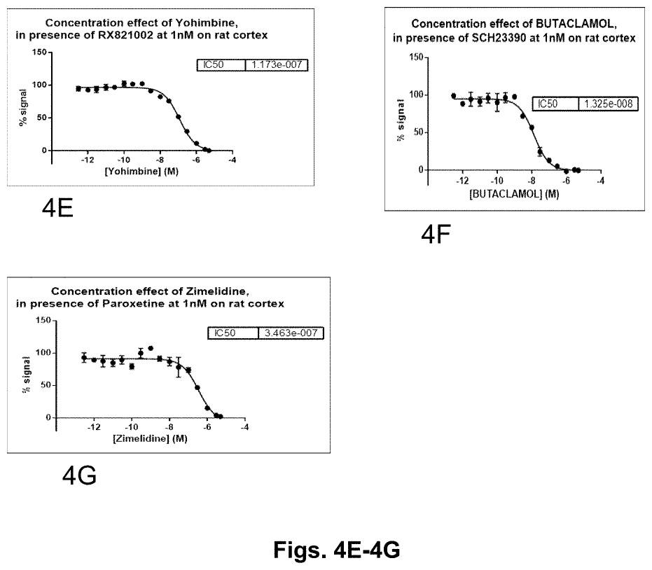

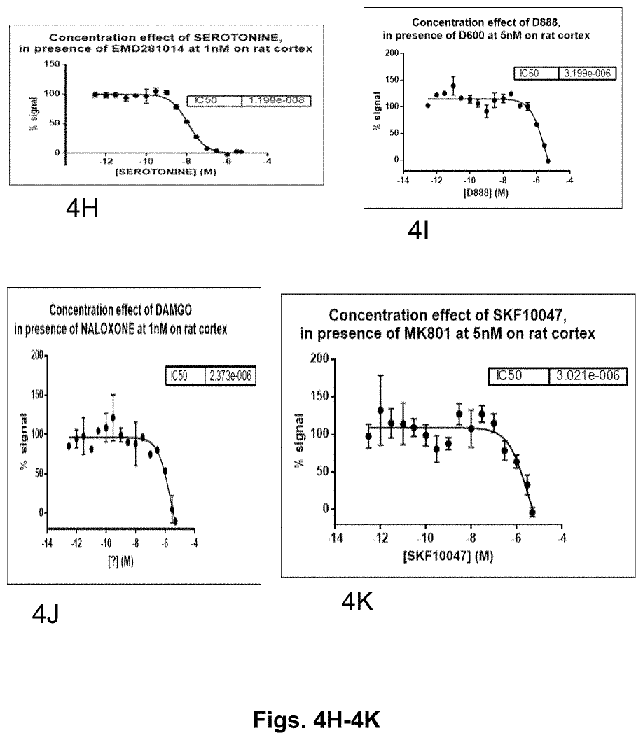

FIGS. 4A-K is series of graphs showing results from a simultaneous binding assay employing rat cortex target molecules. The ligands and test compounds are shown in each figure. The target molecules are the follow receptor molecules: A1 (adenosine receptor) (FIG. 4A); M1 (muscarinic receptor) (FIG. 4B); 5-HT 2A (serotonin receptor) (FIG. 4C); Alpha 1ns (adrenergic receptor) (FIG. 4D); Alpha 2 ns (adrenergic receptor) (FIG. 4E); D1 (dopamine receptor) (FIG. 4F); 5HTtrans (serotonin receptor) (FIG. 4G); 5-HT_{2A} receptor (FIG. 4H); Ca++ channel (FIG. 4I); mu opioid receptor (FIG. 4J); PCP (sigma opioid receptor) (FIG. 4K).

As shown in FIGS. 4A-K, 11 different target molecules were studied simultaneously. Different tissues may be used. For example, the first target receptor molecule, adenosine receptor A1 is also found in smooth muscle throughout the vascular system.

The experiment in the FIGS. 4 A-K may be summarized as follows (Table 7):

TABLE 7

| FIG. | Target molecule | Marker ligand | Test compound |
|---|---|---|---|
| 4A | Adenosine receptor A1 | CPX | NECA |
| 4B | Muscarinic | pyrenzepine | Atropine |
| | acetylcholine receptor | | |
| 4C | 5-HT_{2A} (serotonin) | 8-OH-DPAT | Serotonin |
| 4D | Alpha-1A adrenergic | prazosine | WB4101 |
| | receptor | | |
| 4E | Alpha-2A adrenergic | RX82102 | Yohmbine |
| | receptor | | |
| 4F | Dopamine receptor D1 | SCH23390 | Butaclamol |
| 4G | 5HT transporter | paroxetine | Zimeldine |
| 4H | 5-HT (serotonin) | EMD281014 | Serotonin |
| 4I | Ca++ channel | D600 | D888 |
| 4J | Opioid receptor | naloxone | DAMGO |
| 4K | PCP (Sigma type | MK801 | SKF10047 |
| | opioid receptor) | | |

### Materials and Methods:

### Rat Cortex Membrane Preparation

Rat cortexes from wister male rats were harvested and transferred to 50 mM Tris-HCl (pH, 7.4) and homogenized by a polyton. The homogenate was centrifuged 50 000 g for 15 minutes at 4° C. The resultant pellet was washed in lyses buffer containing 50 mM Tris-HCl (pH, 7.4) containing 1 μg/m1Leupeptin and 1 μM Pepstatin and was centrifuged 50 000 g for 15 minutes at 4° C. The pellet was finally resuspended in a smaller volume of lyses buffer and the final protein concentration was determined according to the Bradford method using bovine serum albumin as a standard.

### Filtration and Elution of Samples

Incubation was terminated by filtration after transfer of the binding sample (aliquot of 200 μl per well) onto 96-well glass filter plates and subsequently filtered rapidly under vacuum the membrane fraction bound to the filters were rinsed several times with wash buffer (50 mM Tris-HCl and 150 mM NaCl) on a vacuum manifold. Membrane filters were pretreated for 1 hour with 50 mM Tris/HCl and 0.3% of Polyethyleneimine solution (PEI).

The filters were dried for one hour at 50° C. and cooled to room temperature before elution of specific ligands using a acetonitrile (contained 100 μM of antipyrine as an internal standard) via a vacuum manifold. Relative quantification of ligand in each sample was performed by UHPLC-MS-MS, the ratio area of ligand and internal standard was used.

### UHPLC-MS/MS Method Development

UHPLC-QQQ analysis was performed by a 1290 Infinity Binary LC system (Agilent Technologies, Waldbronn, Germany) coupled to a Q-TRAP 5500 mass spectrometer with an ESI Turbo V ion source (SCIEX, Foster City, Calif., USA).

Chromatographic separation was performed on C₁₈ column (Poroshell 120 EC-C18, Agilent). The injection volume was 20 μl (full loop injection). The mobile phase consisted of two solutions including solvent A (0.1% formic acid and 6 mM ammonium acetate in water) and solvent B (0.1% formic acid and 6 mM ammonium acetate in acetonitrile), the column was thermostated in an oven at 35° C. and the flow rate was 650 μl/min.

The chromatographic gradient used for C18 column; initial composition of B was 0% during 0.3 min and increased to 80% from 0.3 to 0.9 min then 100% was reached at 1 min until 1.3 min, followed by re-equilibration to initial condition during 0.3 min.

For MS analysis, data were acquired using electrospray ionization (ESI) in positive mode, the Ion Spray Voltage was set at 5 500 V. The desolvation in source was accomplished using the following set parameters: Temperature (TEM) at 600° C., Ion Source Gas 1 (GS1) at 40 PSI, Ion Source Gas 2 (GS2) at 50 PSI, and Curtain Gas (CUR) at 50 PSI. The specific parameters of MRM method which to permit to quantify and monitored the ligands are described in Table 8. Raw Data were processed in Sciex Analyst and individual AUC (area under the curve) for each analyte in each sample was determined using the MultiQuant software.

TABLE 8

| Q1 Mass | Q3 Mass | Time | | DP | EP | CE | CXP |
|---|---|---|---|---|---|---|---|
| (Da) | (Da) | (msec) | ID | (volts) | (volts) | (volts) | (volts) |
| 305.200 | 263.100 | 150 | CPX | 100 | 10 | 32 | 11 |
| 352.200 | 113.000 | 150 | PIRENZEPINE | 80 | 10 | 27 | 18 |
| 384.142 | 95.000 | 150 | PRAZOSINE | 196 | 10 | 77 | 14 |
| 235.100 | 203.100 | 150 | RX821002 | 20 | 10 | 23 | 9 |
| 288.100 | 179.115 | 150 | SCH23390 | 10 | 10 | 31 | 8 |
| 248.100 | 147.100 | 150 | 8-OH-DPAT | 40 | 10 | 28 | 7 |
| 377.200 | 209.200 | 150 | EMD281014 | 20 | 10 | 31 | 9 |
| 330.100 | 192.200 | 150 | PAROXETINE | 40 | 10 | 29 | 8 |
| 485.500 | 165.100 | 150 | D600 | 60 | 10 | 37 | 22 |
| 222.100 | 178.100 | 150 | MK801 | 50 | 10 | 54 | 8 |
| 328.100 | 212.200 | 150 | NALOXONE | 60 | 10 | 53 | 9 |

DP: de-clustering potential, EP: entrance potential, CE: collision energy and CXP: Collision Cell Exit Potential.

### Binding by MS Experiments

### Mass Binding Competitive Assays:

The ligand displacement assays was performed using rat cortex membrane preparations naturally containing the following receptors A1 (adenosine), M1 (muscarinic), Alpha1ns (adrenergic), Alpha2 ns (adrenergic), D1 (dopamine), 5HT1a (serotonin), 5HT2a (serotonin), 5HTtrans (serotonin), Ca²⁺ channel (verapamil site), Glutamate (Non-Selective) Rat Ion Channel, and Opioid non selective receptors.

TABLE 9

| | Specific Ligand/ | |
|---|---|---|
| Receptor | concentration used | Inhibitor |
| A1—adenosine | CPX/1 nM | NECA |
| M1—muscarinic | PIRENZEPINE/1 nM | atropine |
| Alpha1ns—adrenergic | PRAZOSINE/1 nM | WB 4101 |
| Alpha2ns—adrenergic | RX821002/1 nM | Yohimbine |
| D1—dopamine | SCH23390/1 nM | Butaclamol |
| 5HT1a—serotonin | 8-OH-DPAT/5 nM | serotonin |
| 5HT2a—serotonin | EMD281014/1 nM | serotonin |
| 5HTtrans—serotonin | PAROXETINE/1 nM | Zimelidine |
| Cave (Ca channel) | D600/1 nM | D888 |
| PCP—Sigma type opioid | MK801/5 nM | SKF10047 |
| receptor | | |
| Opioid ns | NALOXONE/1 nM | DAMGO |

The ligand displacement assays were performed using 8 concentrations of the inhibitor (see Table 9) (in a range from 0.1 nM to 100 μM) and a mixture of a single concentration of each specific ligand (see Table 9). They were co-incubated with 200 μg/well of rat membrane cortex in assay buffer (50 mM Tris-HCl, 5 mM EDTA/Tris, 150 mM NaCl, 5 mM KCl, 2 mM MgCl2 and 0.1% BSA), in triplicate. Incubation was terminated by filtration after incubation for 60 minutes at 22° C. The remaining quantity of each specific ligand (see Table 9) was determined by UHPLC-MS/MS.

### Example 5: Multiplexing Different, Heterologous Tissues—Ex Vivo Membranes: Rat Cortex, Rat Cerebellum and Rat Ventricular Tissue

This example shows multiplexing an MS competing binding assay as described, but different tissues in the same experiment.

Results are shown in the Table 10 below. Different tissues are used in this example. Exemplary tissue sources for target molecule receptors are cerebral cortex, cerebellum, and ventricular membrane (rat or human). The binding assays shown in column 1 were A1, M1, etc. In each case, a known ligand (shown as [ligand] in column 2) was added and the extent of binding to the tissues studied was measures. The known (marker) ligands were as used in Example 4. A calibration curve was prepared. As shown below, SNR indicates signal to noise and % CV indicates percent coefficient of variation.

TABLE 10

| | | | | ventricular |
|---|---|---|---|---|
| Binding | | rat cortex | cerebellum | (membrane) |
| assay | | 180 μg | 180 μg | 180 μg |
| A1 | [ligand]: nM | 0.1 | 1 | 5 |
| | SNR: | 7 | 4.5 | 1.5 |
| | % CV | 5.30 | 3.7 | 51.6 |
| M1 | [ligand]: nM | 5 | | |
| | SNR: | 17.5 | | |
| | % CV | 6.5 | | |
| Alpha 1ns | [ligand]: nM | 0.1 | 0.1 | 0.1 |
| | SNR: | 53.7 | 13.5 | 28.1 |
| | % CV | 12 | 25.7 | 13.1 |
| Alpha 2 ns | [ligand]: nM | 0.1 | 1 | |
| | SNR: | 51.3 | 11.2 | |
| | % CV | 4.2 | 6.5 | |
| D1 | [ligand]: nM | 1 | | |
| | SNR: | 46.6 | | |
| | % CV | 7.7 | | |
| 5HT1a | [ligand]: nM | 1 | | |
| | SNR: | 4.1 | | |
| | % CV | 32.7 | | |
| 5HT2a | [ligand]: nM | 0.1 | 0.1 | 1 |
| | SNR: | 14.2 | 2.5 | 2.3 |
| | % CV | 12.2 | 80.4 | 113.4 |
| 5HT trans | [ligand]: nM | 0.1 | 0.1 | 0.1 |
| | SNR: | 5 | 2.5 | 2.8 |
| | % CV | 6.9 | 29.4 | 127.5 |
| CAVE | [ligand]: nM | 1 | 0.1 | |
| | SNR: | 2.4 | 3.2 | |
| | % CV | 7 | 31.7 | |
| PCP | [ligand]: nM | | 10 | 50 |
| | SNR: | | 2.2 | 2.4 |
| | % CV | | 26.9 | 27.4 |
| OPIOID ns | [ligand]: nM | 1 | | |
| | SNR: | 9.8 | | |
| | % CV | 8.7 | | |

### Example 6: Multiplexing in a Single Well—Mass Binding of 20 Ligands in Mixtures of Rat Ex Vivo Membranes or Mixtures of Recombinant Membranes

In this example, different tissues and/or receptor molecules are combined in the same well in a single reaction. Rat cortex, cerebellum, and ventricular membrane are added to a single well and a series of reaction are carried out, using ligands as shown in Example 5. Materials and methods:

### Ex Vivo Membrane Preparation

Rat cortexes from wister male rats are harvested and transferred to 50 mM Tris-HCl (pH, 7.4) and homogenized by a polyton. The homogenate was centrifuged 50 000 g for 15 minutes at 4° C. The resultant pellet is washed in lyses buffer containing 50 mM Tris-HCl (pH, 7.4) containing 1 μg/ml Leupeptin and 1 μM Pepstatin and is centrifuged 50 000 g for 15 minutes at 4° C. The pellet is finally resuspended in a smaller volume of lyses buffer and the final protein concentration is determined according to the Bradford method using bovine serum albumin as a standard.

Rat cerebellum, hepatic and ventricular membrane preparations are performed as described above.

### Recombinant Membrane Preparation

### Cell Culture and Expression

A stable transfection of a human cell line is performed using suitable expression vector containing the coding sequences for the receptor of interest. Single colonies of stably transfected cells are further cultivated in selection media using a specific antibiotic. Final clone selection is based on binding affinities of clones for a specific ligand.

### Membrane Extraction

A dry cell pellet of a clone of a human cells stably expressing the receptor of interest was resuspended in lysis buffer (50 mM Tris-HCl, 5 mM Tris-EDTA, 20 mM NaCl, 1.5 mM CaCl2, 5 mM MgCl2, 10 μg/ml trypsin inhibitor, 1 μg/ml leupeptin, 75 μg/ml PMSF). The cells are lysed using an ultrasonic probe (Sonifier 250, Branson). The cell lysate is centrifuged at 50 000 xg for 15 minutes at 4° C. The membrane pellet is resuspended in lysis buffer containing 10% (v/v) glycerol and the final protein concentration is determined according to the Bradford method using bovine serum albumin as a standard.

### Filtration and Elution of Samples

Incubation is terminated by filtration after transfer of the binding sample (aliquot of 200 μl per well) onto 96-well glass filter plates and subsequently filtered rapidly under vacuum the membrane fraction bound to the filters are rinsed several times with wash buffer (50 mM Tris-HCl and 150 mM NaCl) on a vacuum manifold. Membrane filters are pretreated for 1 hour with 50 mM Tris/HCl and 0.3% of Polyethyleneimine solution (PEI).

The filters are dried for one hour at 50° C. and cooled to room temperature before elution of specific ligands using a acetonitrile (contained 100 μM of antipyrine as an internal standard) via a vacuum manifold. Relative quantification of ligand in each sample is performed by UHPLC-MS-MS, the ratio area of ligand and internal standard is used.

### UHPLC-MS/MS Method Development

UHPLC-QQQ analysis is performed by a 1290 Infinity Binary LC system (Agilent Technologies, Waldbronn, Germany) coupled to a Q-TRAP 5500 mass spectrometer with an ESI Turbo V ion source (SCIEX, Foster City, Calif., USA).

Chromatographic separation is performed on C₁₈ column (Poroshell 120 EC-C18, Agilent). The injection volume is 20 μl (full loop injection). The mobile phase consisted of two solutions including solvent A (0.1% formic acid and 6 mM ammonium acetate in water) and solvent B (0.1% formic acid and 6 mM ammonium acetate in acetonitrile), the column is thermostated in an oven at 35° C. and the flow rate is 650 μl/min.

The chromatographic gradient used for C₁₈ column; initial composition of B is 0% during 0.3 min and increased to 80% from 0.3 to 0.9 min then 100% is reached at 1 min until 1.3 min, followed by re-equilibration to initial condition during 0.3 min.

For MS analysis, data are acquired using electrospray ionization (ESI) in positive mode, the Ion Spray Voltage is set at 5 500 V. The desolvation in source is accomplished using the following set parameters: Temperature (TEM) at 600° C., Ion Source Gas 1 (GS1) at 40 PSI, Ion Source Gas 2 (GS2) at 50 PSI, and Curtain Gas (CUR) at 50 PSI. The specific parameters of MRM method which to permit to quantify and monitored the ligands is described in Table 11. Raw Data are processed in Sciex Analyst and individual AUC (area under the curve) for each analyte in each sample is determined using the MultiQuant software.

TABLE 11

| Q1 Mass | Q3 Mass | Time | | DP | EP | CE | CXP |
|---|---|---|---|---|---|---|---|
| (Da) | (Da) | (msec) | ID | (volts) | (volts) | (volts) | (volts) |
| 305.200 | 263.100 | 50 | CPX | 100 | 10 | 32 | 11 |
| 408.1 | 219.2 | 50 | CGS 21680 | 131 | 10 | 35 | 10 |
| 300.2 | 270.2 | 50 | AB-MECA | 175 | 10 | 19 | 13 |
| 352.200 | 113.000 | 50 | PIRENZEPINE | 80 | 10 | 27 | 18 |
| 479.3 | 240.1 | 50 | AF-DX 384 | 120 | 10 | 28 | 9 |
| 384.142 | 95.000 | 50 | PRAZOSINE | 196 | 10 | 77 | 14 |
| 235.100 | 203.100 | 50 | RX821002 | 20 | 10 | 23 | 9 |
| 288.100 | 179.115 | 50 | SCH23390 | 10 | 10 | 31 | 8 |
| 356.2 | 325.2 | 50 | Methylspiprone | 90 | 10 | 15 | 13 |
| 248.100 | 147.100 | 50 | 8-OH-DPAT | 40 | 10 | 28 | 7 |
| 377.200 | 209.200 | 50 | EMD281014 | 20 | 10 | 31 | 9 |
| 330.100 | 192.200 | 50 | PAROXETINE | 40 | 10 | 29 | 8 |
| 485.500 | 165.100 | 50 | D600 | 60 | 10 | 37 | 22 |
| 222.100 | 178.100 | 50 | MK801 | 50 | 10 | 54 | 8 |
| 328.100 | 212.100 | 50 | NALOXONE | 60 | 10 | 53 | 9 |
| 1052.5 | 958.2 | 50 | CGP 42112A | 50 | 10 | 17 | 10 |
| 1060.6 | 938.5 | 50 | Bradykinine | 75 | 10 | 22 | 16 |
| 352.200 | 113.000 | 50 | CP 55,940 | 80 | 10 | 27 | 18 |
| 1064.2 | 1001.2 | 50 | CCK8 | 76 | 10 | 23 | 8 |
| 112.1 | 95.1 | 50 | Histamine | 159 | 10 | 24 | 11 |
| 497.3 | 434.3 | 50 | LTD4 | 125 | 10 | 26 | 17 |

DP: declustering potential, EP: entrance potential, CE: collision energy and CXP: Collision Cell Exit Potential.

### Binding by MS Experiments

### MASS Binding Competitive Assays:

The ligand displacement assays are performed using mixtures of 4 different ex vivo membranes of rat cortex, cerebellum, ventricular and hepatic membrane preparations. An equal quantity of each tissue membrane preparation is mixed (50 μg).

Additionally, ligand displacement assays are also performed using a mixture of 20 different recombinant membranes (see Table 12), equal quantities (10 μg) of each membrane preparation is mixed.

TABLE 12

| Receptor | Specific Ligand | Inhibitor |
|---|---|---|
| A1 | CPX | NECA |
| A2A (h) | CGS 21680 | NECA |
| A3 (h) | AB-MECA | IB-MECA |
| M1 | PIRENZEPINE | atropine |
| M2 (h) | AF-DX 384 | 4-DAMP |
| Alpha1ns | PRAZOSINE | WB 4101 |
| Alpha2ns | RX821002 | Yohimbine |
| D1 | SCH23390 | Butaclamol |
| D2S (h) | Methylspiprone | Butaclamol |
| 5HT1a | 8-OH-DPAT | serotonin |
| 5HT2a | EMD281014 | serotonin |
| 5HTtrans | PAROXETINE | Zimelidine |
| Cave | D600 | D888 |
| PCP | MK801 | SKF10047 |
| Opioid ns | NALOXONE | DAMGO |
| AT2 (h) | CGP 42112A | Angiotensine II |
| B2 (h) | Bradykinine | HOE 140 |
| CB1 (h) | CP 55,940 | WIN 55,212-2 |
| CCK1 (CCKA) | CCK-8S | SIB-CCK8 |
| H4 (h) | Histamine | PDGF-BB |
| CysLT1 (LTD4) (h) | LTD4 | MK571 |

Mass binding competitive assays are performed using 8 concentrations of the inhibitors (see table 12) (in a range from 0.1 nM to 100 μM) and a mixture of a single concentration (of each specific ligand (see table 12) in which each ligand is at a final concentration of 5 nM. They are co-incubated in 200 μg/well of either the ex vivo membrane mixture or a recombinant membrane mixture in assay buffer (50 mM Tris-HCl, 5 mM EDTA/Tris, 150 mM NaCl, 5 mM KCl, 2 mM MgCl2 and 0.1% BSA), in triplicate. Incubation is terminated by filtration after incubation for 60 minutes at 22° C. The remaining quantity of each specific ligand (see table) is determined by UHPLC-MS/MS.

### Example 7: Multiplexing in a Single Well for Safety Testing

In this example, a combination of different tissue types is combined in individual wells as shown in the Table 13 below:

TABLE 13

| Receptor | Tissue | Known Ligand/substrate |
|---|---|---|
| GPCR, Adenosine | ubiquitous throughout the | Adenosine |
| receptor A1 | entire body. | |
| cyclooxygenase 2 | synoviocytes, endothelial | Arachidonic acid |
| (COX2) | cells, chondrocytes, | |
| | osteoblasts, and | |
| | monocytes/macrophages, | |
| | stimulated with cytokines | |
| Monoamine | Hypothalamus and | Serotonin, melatonin, |
| oxidase (MAO) | hippocampal uncus | norepinephrine, |
| | | epinephrine |
| Dopamine | Brain (substantia nigra) | dopamine |
| transporter | | |

The above target receptor molecules can be obtained from the listed tissue or produced in a cloned cell.

Materials and methods are carried out as described above.

### Example 8A, 8B: Pharmacology Kₒₙ and K_{off} Determination

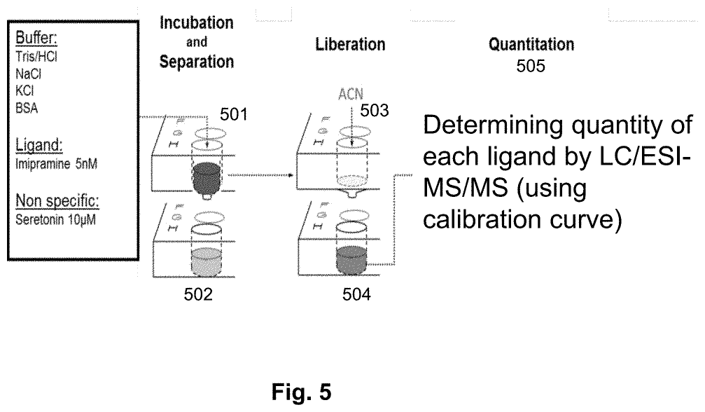

FIG. 5 is a schematic workflow for using MS to determine binding kinetics of a test compound to its cognate receptor molecule. As shown, material containing target molecules (e.g. rat cortex) is incubated with a ligand and a test compound (in this figure imipramine and serotonin). The bound ligands are recovered by methods as described above and the quantity of each ligand is determined. As shown in the FIG. 5, a sample comprising at least one target receptor molecule (e.g. rat cortex) is first incubated 501 with a ligand and a test compound e.g. imipramine (5 nM) and Serotonine (10 μM) respectively in a buffer comprising Tris/HCl, NaCl, KCl, and BSA. Following incubation, the bound receptor-ligand complex is separated 502 by methods as described in the present invention. The separated ligand-receptor complex is further treated so as to separate the ligand from the bound target receptor molecule e.g. by the use of acetonitrile and a glass filter which allows passage only of unbound ligand (503). Following recovery of bound ligand molecules, from each well of a multiple plate reader 504 is quantitated 505 by liquid. chromatography/ESI-MS/MS using calibration curve to determine Kₒₙ and K_{off}.

As shown in FIG. 5, buffer, a ligand (imipramine) and the non-specific binder serotonin are incubated in various wells. Separation of the complexed target molecule in rat cortex, serotonin transporter (5-HT) is carried out as before. The complex is separated using acrylonitrile and the separated serotonin is measured by MS to determine Kₒₙ and K_{off}.

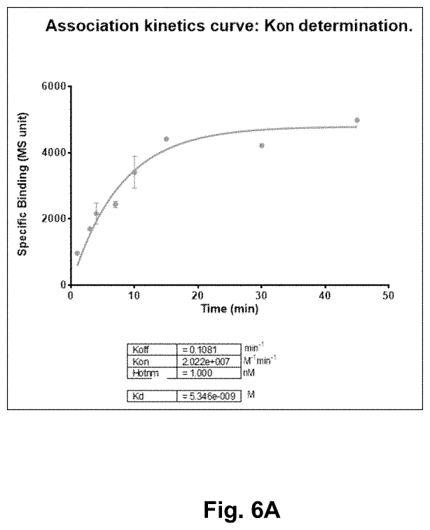

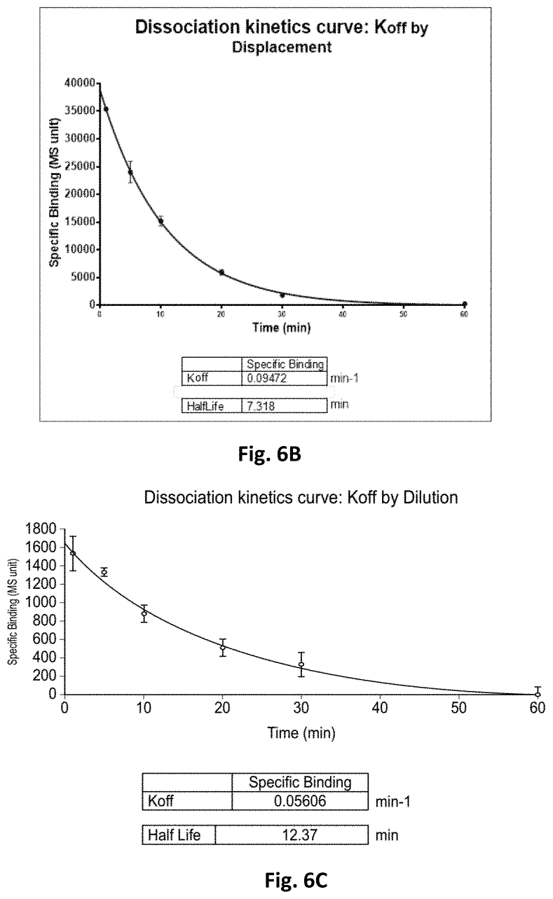

FIGS. 6A, 6B, and 6C is a series of graphs showing results of an MS method to determine association kinetics, Kₒₙ (FIG. 6A) and dissociation kinetics, K_{off} (FIGS. 6B and 6C). In FIG. 6B, dissociation kinetics of GABAB1_{b/2} from CGP54626, at a concentration of 1 nM by the displacement approach via the addition of 10 μM. CPG52432. Data points represent specific binding (means+/−SD, n=2). In FIG. 6C, shows dissociation kinetics of GABAB1_{b/2} from CGP54626 at a concentration of 5 nM by the dilution approach. Data points represent specific binding (means+/−SD, n=2).

FIG. 6A shows the association kinetics curve and Kₒₙ determination by measuring specific binding at different time intervals. FIG. 6B shows the dissociation kinetics curve and K_{off} obtained by measuring the decrease of specific binding of the ligand to the target receptor molecule over time. FIG. 6C shows dissociation kinetics as measured by dilution.

### Example 8A: GABA 1b Kₒₙ/K_{off}

### Cell Culture and Expression of GABA_{B1b/2}

A stable transfection of CHO—S cell line was performed using the pCi/neo vector (Promega) containing the coding sequences for the human GABA B receptor consisting of 2 units 1b (NM_021903) as well as GABA 2 (NM_005458). Single colonies of stably transfected cells were further cultivated in selection media using geneticin. Final clone selection was based on binding affinities of clones for ^{3H}[CGP54626].

### Membrane Extraction

A dry cell pellet of a clone of a CHO—S cells stably expressing GABA_{B1b/2} resuspended in lysis buffer (50 mM Tris-HCl, 5 mM Tris-EDTA, 20 mM NaCl, 1.5 mM CaCl2, 5 mM MgCl₂, 10 μg/ml trypsin inhibitor, 1 μg/ml leupeptin, 75 μg/ml PMSF). The cells were lysed using an ultrasonic probe (Sonifier 250, Branson). The cell lysate was centrifuged at 50 000 xg for 15 minutes at 4° C. The membrane pellet was resuspended in lysis buffer containing 10% (v/v) glycerol and the final protein concentration was determined according to the Bradford method using bovine serum albumin as a standard.

### Filtration and Elution of Samples

Incubation was terminated by filtration after transfer of the binding sample (aliquot of 200 μl per well) onto 96-well glass filter plates and subsequently filtered rapidly under vacuum the membrane fraction bound to the filters were rinsed several times with wash buffer (50 mM Tris-HCl and 150 mM NaCl) on a vacuum manifold. Membrane filters were pretreated for 1 hour with 50 mM Tris/HCl and 0.3% of Polyethyleneimine solution (PEI).

The filters are dried for one hour at 50° C. and cooled to room temperature before elution of CGP54626 using a acetonitrile (contained 100 μM of antipyrine as an internal standard) via a vacuum manifold. Relative quantification of ligand in each sample was performed by UHPLC-MS-MS, the ratio area of ligand and internal standard was used.

### UHPLC-MS/MS Method Development

UHPLC-QQQ analysis was performed by a 1290 Infinity Binary LC system (Agilent Technologies, Waldbronn, Germany) coupled to a Q-TRAP 5500 mass spectrometer with an ESI Turbo V ion source (SCIEX, Foster City, Calif., USA).

Chromatographic separation was performed on C₁₈ column (Poroshell 120 EC-C18, Agilent). The injection volume was 20 μl (full loop injection). The mobile phase consisted of two solutions including solvent A (0.1% formic acid and 6 mM ammonium acetate in water) and solvent B (0.1% formic acid and 6 mM ammonium acetate in acetonitrile), the column was thermostated in an oven at 35° C. and the flow rate was 650 μl/min.

The chromatographic gradient used for C18 column; initial composition of B was 0% during 0.3 min and increased to 80% from 0.3 to 0.9 min then 100% was reached at 1 min until 1.3 min, followed by re-equilibration to initial condition during 0.3 min.

For MS analysis, data were acquired using electrospray ionization (ESI) in positive mode, the Ion Spray Voltage was set at 5 500 V. The desolvation in source was accomplished using the following set parameters: Temperature (TEM) at 600° C., Ion Source Gas 1 (GS1) at 40 PSI, Ion Source Gas 2 (GS2) at 50 PSI, and Curtain Gas (CUR) at 50 PSI. The specific parameters of MRM method which to permit to quantify and monitored the ligand (CGP54626) is described in Table 14. Raw Data were processed in Sciex Analyst and individual AUC (area under the curve) for each analyte in each sample was determined using the MultiQuant software.

TABLE 14

| Q1 Mass | Q3 Mass | Time | | DP | EP | CE | CXP |
|---|---|---|---|---|---|---|---|
| (Da) | (Da) | (msec) | ID | (volts) | (volts) | (volts) | (volts) |
| 408.1 | 236.0 | 150 | CGP54626-1 | 131 | 10 | 27 | 10 |
| 408.1 | 219.2 | 150 | CGP54626-2 | 131 | 10 | 35 | 10 |

DP: declustering potential, EP: entrance potential, CE: collision energy and CXP: Collision Cell Exit Potential.

### Binding by MS Experiments

### Optimal Concentration of Receptor and Ligand Determination

Membrane preparations containing GABA_{B1b/2} and CGP54626 were incubated in triplicates in assay buffer (50 mM Tris-HCl, 2.5 mM CaCl2, 10 μg/ml trypsin, 1 μg/ml leupeptin, 1 μg/ml pepstatin) in polypropylene 96-deep-well plates at 22° C. Initially, 6 concentrations (0.1, 0.5, 1, 3, 5, 10, 25 and 50 nM) of CGP54626 (Tocris, ref: 1088) was co-incubated for 60 minutes at 22° C., with 3 concentrations (45, 100 and 180 μg/well) of the recombinant receptor GABA_{B1b/2}.

Non-specific binding was determined by the co-incubation with 10 μM CGP52432.

The incubation was terminated by filtration after transfer of the total volume of the binding reaction to a filter plate. The remaining quantity of CGP54626 was determined by UHPLC-MS/MS.

### For Saturation Assays:

Membrane aliquots containing 10 to 180 μg of GABA_{B1b/2} of protein were incubated in triplicate in the presence of 1 nM of CGP54626 in a total volume of 200 μl of assay buffer. Incubation was terminated by filtration after incubation for 60 minutes at 22° C.

Non-specific binding was determined by the co-incubation with 10 μM CGP52432

The incubation was terminated by filtration after transfer of the total volume of the binding reaction to the filter plate. The remaining quantity of CGP54626 was determined by UHPLC-MS/MS.

Mass binding association assays (Kₒₙ):

Membrane aliquots containing 22.5 μg/100 μl of GABA_{B1b/2} membrane protein were incubated in a total volume of 2000 μl of assay buffer at 22° C. with 1 nM CGP54626. At each time point 200 μl of reaction mix was removed the incubation was terminated by filtration. The remaining quantity of CGP54626 was determined by UHPLC-MS/MS. See FIG. 6A.

Non-specific binding was determined by the co-incubation with 10 μM CGP52432.

### Mass Binding Competitive Assays:

The ligand displacement assays was performed using eight concentrations of the competing ligand, CGP52432 (in a range from 1 nM to 30 μM), GABA (in a range from 10 nM to 1 mM) and baclofen (in a range from 10 nM to 1 mM). They were co-incubated with 45 μg/well of GABA_{B1b/2} membrane protein and 1 nM CGP54626 in assay buffer, in triplicate. Incubation was terminated by filtration after incubation for 60 minutes at 22° C. The remaining quantity of CGP54626 was determined by UHPLC-MS/MS.

### Mass Binding Dissociation Assays—Displacement.

Membrane aliquots containing 22.5 μg/100 μl of GABA_{B1b/2} membrane protein were incubated in a total volume of 2000 μl of assay buffer at 22° C. with 1 nM CGP54626. The reaction was allowed to reach equilibrium for 60 minutes before starting the dissociation via the addition of 10 μM CPG52432. Dissociation was stopped at defined time intervals (1 to 80 minutes) via the filtration of 200 μl of the reaction mix. Samples for each time point were prepared in duplicate. The remaining quantity of CGP54626 was determined by UHPLC-MS/MS. See FIG. 6B.

### Mass Binding Dissociation Assays—Dilution Method

For the determination of the K_{off} constant by dilution 112.5 μg/100 μl of GABA_{B1b/2} membrane protein were incubated with 5 nM CGP54626 at 22° C. for 60 minutes. An aliquot of 22 μl was removed and added to 2178 μl of assay buffer resulting in a 1:100 dilution. Dissociation was stopped by filtration after defined time intervals (1 to 80 minutes). Samples for each time point were prepared in duplicate. The reaming quantity of CGP54626 was determined by UHPLC-MS. See FIG. 6C.

### Example 8B: Binding by Mass Spectrometry Experiments Multiplexing of kₒₙ/k_{off} Determination on Either a Single Ex Vivo Membrane or Mixtures of Ex Vivo Membranes Alternatively on Mixtures of Recombinant Membranes

### Preparation of Membrane Mixtures

The Kₒₙ and K_{off} determinations are performed either on rat cortex membrane or by using mixtures of 4 different ex vivo membranes of rat cortex, cerebellum, ventricular and hepatic membrane preparations. An equal quantity of each tissue membrane preparation is mixed (50 μg). Additionally, Kₒₙ and K_{off} determinations are also performed using a mix of 20 different recombinant membranes (see Table 15), equal quantities of each membrane preparation is mixed (10 μg).

TABLE 15

| Receptor | Specific Ligand | Inhibitor |
|---|---|---|
| A1 | CPX | NECA |
| A2A (h) | CGS 21680 | NECA |
| A3 (h) | AB-MECA | IB-MECA |
| M1 | PIRENZEPINE | atropine |
| M2 (h) | AF-DX 384 | 4-DAMP |
| Alpha1ns | PRAZOSINE | WB 4101 |
| Alpha2ns | RX821002 | Yohimbine |
| D1 | SCH23390 | Butaclamol |
| D2S (h) | Methylspiprone | Butaclamol |
| 5HT1a | 8-OH-DPAT | serotonin |
| 5HT2a | EMD281014 | serotonin |
| 5HTtrans | PAROXETINE | Zimelidine |
| Cave | D600 | D888 |
| PCP | MK801 | SKF10047 |
| Opioid ns | NALOXONE | DAMGO |
| AT2 (h) | CGP 42112A | Angiotensine II |
| B2 (h) | Bradykinine | HOE 140 |
| CB1 (h) | CP 55,940 | WIN 55,212-2 |
| CCK1 (CCKA) | CCK-8S | SIB-CCK8 |
| H4 (h) | Histamine | PDGF-BB |
| CysLT1 (LTD4) (h) | LTD4 | MK571 |

### Mass Binding Association Assays (Kₒₙ):

Membrane aliquots containing 22.5 μg/100 μl of each membrane protein mix are incubated in a total volume of 2000 μl of assay buffer at 22° C. with a mixture of specific ligands (see Table 15) each at a final concentration of 1 nM. At each time point 200 μl of reaction mix is removed the incubation is terminated by filtration. The remaining quantity of each specific ligand (see table) is determined by UHPLC-MS/MS.

Non-specific binding is determined by the co-incubation of a mix of specific inhibitors (see table) each at a final concentration of 10 μM.

### Mass Binding Dissociation Assays—Displacement

Membrane aliquots containing 22.5 μg/100 μl of each membrane protein mix a incubated in a total volume of 2000 μl of assay buffer at 22° C. with a mixture of specific ligands (see Table 15) each at a final concentration of 1 nM. The reaction is allowed to reach equilibrium for 60 minutes before starting the dissociation via the addition of a mixture of specific inhibitors (see table) each at a final concentration of 10 μM. Dissociation is stopped at defined time intervals (1 to 80 minutes) via the filtration of 200 μl of the reaction mix. Samples for each time point are prepared in duplicate. The remaining quantity of each specific ligand was determined by UHPLC-MS/MS.

### Mass Binding Dissociation Assays—Dilution Method

For the determination of the K_{off} constant by dilution 112.5 μg/100 μl of each membrane protein mix are incubated with a mixture of specific ligands (see table) each at a final concentration of 1 nM and incubated at 22° C. for 60 minutes. An aliquot of 22 μl was removed and added to 2178 μl of assay buffer resulting in a 1:100 dilution. Dissociation is stopped by filtration after defined time intervals (1 to 80 minutes). Samples for each time point are prepared in duplicate. The remaining quantity of each specific ligand is determined by UHPLC-MS/MS.

### CONCLUSION

The above specific description is meant to exemplify and illustrate the invention and should not be seen as limiting the scope of the invention, which is defined by the literal and equivalent scope of the appended claims. Any patents or publications mentioned in this specification are intended to convey details of methods and materials useful in carrying out certain aspects of the invention which may not be explicitly set out but which would be understood by workers in the field. Such patents or publications are hereby incorporated by reference to the same extent as if each was specifically and individually incorporated by reference and contained herein, as needed for the purpose of describing and enabling the method or material referred to.

The preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A multiplexed method for quantitating binding of a test compound to a predetermined target molecule and also to binding to off-target target molecules, comprising the steps of:
(a) obtaining a mixture of target molecules from at least one of (i) healthy or non-healthy human or non-human tissue, and (ii) a synthetic protein preparation;
(b) incubating said target molecules in a plurality of mixtures of ligands and test compounds, wherein said target molecules and are incubated with different ligands;
(c) removing unbound ligands from said plurality of mixtures;
(d) isolating ligands that were bound to target molecules in said mixture of target molecules;
(e) determining a quantity of ligand that was bound by a target molecule, by measuring ligands that were obtained in step (d), using mass spectrometry and a calibration curve;
(f) determining an affinity of the test compound for target molecules in said mixture of target molecules using data obtained in step (e); and
(g) measuring binding of said test compound to a predetermined target molecule and comparing said binding to binding of said test compound to off-target molecules.

2. The method of claim 1, wherein said mixture of target molecules further comprises a heterologous mixture of target molecules.

3. The method of claim 1, wherein said mixture of target molecules comprises targets that are human target molecules.

4. (canceled)

5. The method of claim 1, wherein step (a) comprises obtaining target molecule from a crude extract.

6. (canceled)

7. (canceled)

8. The method of claim 1, further comprising the step of determining a Kₒₙ and K_{off} of the test compound to a target molecule.

9. The method of claim 8, wherein K_{off} is determined by a displacement method.

10. The method of claim 8, wherein K_{off} is determined by a dilution method.

11. The method of claim 1, wherein said target molecules are formed in a mixture of receptor target molecules that does not exist in nature in a single mixture.

12. (canceled)

13. (canceled)

14. A multiplexed method for quantitating binding affinity of at least two different test compounds (test compound C1-Cₙ) to at least two different receptor target molecules (receptor RT1 for C1, RTₙ for Cₙ), based on competitive binding between the test compounds and known binders for RT1 and RT2 (known binder B1-Bₙ), comprising:
(a) providing a mixture comprising (i) test compounds C1-Cₙ; (ii) known binders B1-Bₙ and (iii) receptor target molecules RT1-RTₙ;
(b) allowing complexes to form in said mixture between the test compounds C1-Cₙ, RT1-RTₙ, and B1-Bₙ,
(c) separating compounds which do not form complexes with their target molecules from said complexes;
(d) isolating known binders from complexes obtained in step (c) and passing isolated binders through a mass spectrometer to measure binding of test compounds using mass spectroscopy; and
(e) determining the relative affinities of compounds C1-Cₙ for RT1-RTₙ, respectively, wherein Cₙ, Bₙ, and RTₙ represent a series of members wherein n is between 2 and 40.

15. The method of claim 14, wherein the receptor target molecules RT1-RTₙ are in a mixture not found in nature in the same tissue.

16. The method of claim 14, wherein step (a) comprises obtaining receptor target molecules from a crude extract and said receptor target molecules are obtained from ex vivo membranes of at least two of cortex, cerebellum, ventricular and hepatic membrane preparations.

17. The method of claim 14, wherein said step of providing receptor target molecules RT1-RTₙ comprises providing human receptor target molecules.

18. (canceled)

19. (canceled)

20. (canceled)

21. The method of claim 14, further comprising the step of determining a Kₒₙ and K_{off} of the test compound to the target molecule.

22. A multiplexed method for quantitating binding affinity of a test compound to a target molecule, comprising the steps of:
(a) obtaining at least three target molecules as set forth in the chart below
Target molecule
Adenosine receptor A1
Muscarinic
acetylcholine receptor
5-HT2A (serotonin)
Alpha-1A adrenergic
receptor
Alpha-_{2A} adrenergic
receptor
Dopamine receptor D1
5HT transporter
5HT1a receptor
5HT2a receptor
Cave Ca channel
PCP receptor
Opioid receptor
(b) incubating said target molecules in a plurality of mixture of ligands and test molecules,
(c) removing unbound ligands from the mixtures;
(d) isolating ligands that were bound to the target molecules after incubating;
(e) determining the quantity of each ligand that was present on the target molecules by measuring ligands that were obtained in step (d) by mass spectrometry, using a calibration curve prepared with known concentrations of ligand; and
(f) calculating an affinity of the test compound for the target molecule from the data obtained in step (e).

23. The method of claim 22, wherein the same test compound is used with each target molecule.

24. The method of claim 22, comprising the use of the following target molecules and ligands:

| Target molecule | Ligand |
|---|---|
| Adenosine receptor A1 | CPX |
| Muscarinic | pyrenzepine |
| acetylcholine receptor | |
| 5-HT_{2A} (serotonin) | EMD281014 |
| Alpha-1A adrenergic | prazosine |
| receptor | |
| Alpha-2A adrenergic | RX82102 |
| receptor | |
| Dopamine receptor D1 | SCH23390 |
| 5HT transporter | paroxetine |
| 5HT1a receptor | 8-OH-DPAT |
| 5HT2a receptor | EMD281014 |
| Cave Ca channel | D600 |
| PCP receptor | MK801 |
| Opioid receptor | naloxone |

25. A multiplexed method for determining Kₒₙ and/or K_{off} values of a of a test compound to a target molecule, comprising the steps of:
(a) obtaining a mixture of target molecules from at least one of (i) healthy or non-healthy human or non-human tissue, and (ii) a synthetic protein preparation;
(b) incubating said target molecules in a plurality of mixtures of ligands and test compounds, wherein said target molecules bind to different ligands and are incubated with different target molecules;
(c) removing unbound ligands from the mixtures;
(d) isolating bound ligands that were bound to the target molecules;
(e) determining a quantity of ligand that was bound by a target molecule, by measuring ligands that were obtained in step (d) at defined time points in a reaction mixture, using mass spectrometry and a calibration curve; and
(f) calculating Kₒₙ or K_{off} of the test compound for the target molecule using data obtained in step (e).

26. The method of claim 25, wherein Kₒₙ and K_{off} are determined in mixtures of different ex vivo membranes comprised of at least two of: cortex, cerebellum, ventricular and hepatic membrane preparations.

27. The method of claim 25, wherein membrane mixtures comprise at least two of receptor A1, A2A (h), A3 (h), M1, M2 (h), Alpha1ns, Alpha2ns, D1, D2S (h), 5HT1a, 5HT2a, 5HTtrans, Cave, PCP, Opioid ns, AT2 (h), B2 (h), CB1 (h), CCK1 (CCKA), H4 (h), and CysLT1 (LTD4) (h).

28. The method of claim 27, wherein the membrane mixtures comprise all of the listed receptors.

29. The method of claim 25, wherein K_{off} is determined by a displacement method.

30. The method of claim 25, wherein K_{off} is determined by a dilution method.
